(19) United States
(12) Patent Application Publication
(10) Pub. No.: US 2006/0178297 A1
(43) Pub. Date: Aug. 10, 2006

(54) Systems and methods for silencing expression of a gene in a cell and uses thereof

(76) Inventors: Carol M. Troy, Hastings-on-Hudson, NY (US); Lloyd A. Greene, Larchmont, NY (US)

Correspondence Address: BAKER & BOTTS, 30 ROCKEFELLER PLAZA, 44TH FLOOR, NEW YORK, NY, 10112, US

(21) Appl. No.: 11/136,344

(22) Filed: May 23, 2005

Related U.S. Application Data
(63) Continuation-in-part of application No. 10/353,902, filed on Jan. 28, 2003.

(51) Int. Cl. A61K 48/00; C12N 5/08; C12N 15/87

(52) U.S. Cl. 514/7; 514/44; 435/455; 435/368

(57) ABSTRACT

The present invention provides RNA-based systems that are capable of silencing expression of a gene in a cell. Also provided are pharmaceutical compositions, cells, and kits that include the systems; cultures of primary cells that have been contacted with the systems; use of the systems in methods of studying protein function in one or more cells; and use of the systems in methods of studying interactions between neurons in culture; and use of the systems in a method of studying the function of mRNA. The present invention further provides methods for silencing expression of a gene in a cell, and methods for determining the function of a gene in a cell. Additionally, the present invention provides systems for use in genetic screening, and methods for performing genetic screening.

### CROSS-REFERENCE TO RELATED APPLICATIONS

This application is a continuation-in-part of U.S. patent application Ser. No. 10/353,902, filed on Jan. 28, 2003, the contents of which are hereby incorporated by reference thereto.

### STATEMENT OF GOVERNMENT INTEREST

This invention was made with government support under NIH Grant Nos. 1 RO1 NS43089 and 1 R29 NS35933. As such, the United States government has certain rights in this invention.

### BACKGROUND OF THE INVENTION

Researchers have discovered a growing number of ribonucleic acids (RNAs) that do not function as messenger RNAs, transfer RNAs, or ribosomal RNAs. These so-called “non-coding” RNAs include a wide variety of RNAs of incredibly diverse function, ranging from the purely structural to the purely regulatory (Riddihough, G., The other RNA world. Science, 296: 1259, 2002). Representative non-coding RNAs include small nuclear RNAs, small nucleolar RNAs, micro-RNAs (e.g., small temporal RNAs or short temporal RNAs (stRNAs)), and short hairpin RNAs (shRNAs). The non-coding RNA that has generated the most interest, however, is the small interfering RNA (siRNA). siRNA is associated with the phenomenon of RNA interference (RNAi), in which double-stranded RNA (dsRNA), when introduced into a cell, silences the expression of a homologous gene within the cell—i.e., the introduced dsRNA “interferes” with gene expression.

The phenomenon of RNAi is ubiquitous among bacteria, fungi, plants, and animals, although the precise mechanism of interference may differ. In eukaryotes, the current model of the RNAi mechanism involves both an initiation and an effector step. In the initiation step, a processing enzyme cleaves the introduced dsRNA into small interfering RNAs (siRNAs) of approximately 21 nucleotides, with 2-nucleotide 3′ overhangs. In the effector step, each siRNA is unwound and incorporated into an RNA-induced silencing complex (RISC), comprising a helicase, an exonucleolytic nuclease, and an endonucleolytic nuclease. Once incorporated into the RISC, the siRNA serves as a guide molecule, directing the RISC to the homologous mRNA transcript for degradation (Hammond et al., Post-transcriptional gene silencing by double-stranded RNA. Nature Rev. Gen., 2:110-19).

RNAi presents an invaluable tool for functional genomics, and for the identification of gene-specific therapies, because it allows researchers to create numerous silenced phenotypes for the purpose of determining the function of a targeted gene. RNAi offers a number of advantages over antisense technology—the approach most frequently cited for achieving post-transcriptional gene silencing. For example, RNAi methods are more effective and more economical than methods involving antisense nucleic acids.

Known methods of delivering ssRNA and dsRNA to cells primarily involve transfection. (For general transfection protocols, see Elbashir et al., Duplexes of 21-nucleotide RNAs mediate RNA interference in mammalian cell culture. Nature, 411:494-98, 2001; and Elbashir et al., RNA interference is mediated by 21 and 22 nt RNAs. Genes & Dev., 15:188-200, 2001). The efficiency of transfection depends on cell type, passage number, and the confluency of the cells. Since cellular uptake of unmodified antisense nucleic acid is very inefficient, a large amount of antisense nucleic acid needs to be synthesized and applied in order to achieve and maintain a sufficient concentration in the target cells (i.e., at or above the level of the endogenous target mRNA). Therefore, a successful antisense strategy requires the introduction of large amounts of single-stranded antisense nucleic acid (DNA or RNA) into cells. In contrast, the cellular uptake of double-stranded RNA is more efficient, thereby permitting RNAi to occur with much smaller amounts of dsRNA. Even so, a need exists for improved dsRNA uptake into the cell.

Cell cultures also provide invaluable tools for functional genomics and the identification of gene-based therapies. In particular, genetic manipulations of cultured cells have provided great insight into the biology of normal and diseased states, by allowing the molecular dissection of important biological pathways. Transgenic animals are a valuable source of genetically-altered primary cells, but creation of knockouts is time-intensive and labor-intensive, and knocked-out proteins can be compensated for during development. Primary cell cultures are particularly attractive to researchers, because they are so similar to living systems. However, in their attempts to study RNAi in neuron cultures, researchers have met with special challenges. In particular, despite evidence of the importance of mRNAs in neuronal development, neurons have proven refractory to the artificial induction of RNAi—even in Caenorhabditis elegans, where systemic and heritable RNAi can be induced by feeding of long, homologous double-stranded RNA (Timmons, Court, & Fire, Ingestion of bacterially expressed dsRNAs can produce specific and potent genetic interference in Caenorhabditis elegans. Gene, 263(1-2):103-12, 2001).

Although RNAi does function in neurons (Krichevsky and Kosik, RNAi functions in cultured mammalian neurons. Proc. Natl. Acad. Sci. USA, 99(18):11926-9 2002; Gaudilliere Shi Bonni, RNA interference reveals a requirement for myocyte enhancer factor 2A in activity-dependent neuronal survival. J. Biol. Chem., 277(48): 46442-6, 2002), its use is still dependent on transfection. Reported transfection efficiencies in neurons are typically very low, and are usually expressed as a fraction of cells surviving the trauma of transfection. Since neurons are post-mitotic, these low efficiencies cannot be countered by establishing stable cell lines. The low transfection rate in neurons means that only gross changes in protein or message-levels can be detected across populations of neurons; it also limits the usefulness of RNAi for researchers who study interactions between neurons in culture (e.g., electrical signaling in neuronal networks, cell-surface-protein expression, and secreted-protein expression). The toxicity of the reagents is of particular concern in heterogeneous primary neuronal cultures, since variation in survival rates across cell types could alter the very system under study. Furthermore, the requirement that cells be given time to recover from transfection (typically 24-48 h) means that little information is available concerning the earliest effects of the manipulations performed, or, indeed, of the RNAi mechanism itself. Accordingly, there also exists in the art a need for methods of achieving artificial RNAi induction in cultured cells, and, in particular, for methods of improving dsRNA uptake into cultured neurons.

Low transfection efficiency is the most frequent cause of unsuccessful silencing in RNAi. Other techniques for dsRNA uptake include electroporation, injection, liposome-facilitated transport, and microinjection. Although direct microinjection of dsRNA into cells is generally considered to be the most effective means known for inducing RNAi, the characteristics of this technique severely limit its practical utility. In particular, direct microinjection can only be performed in vitro, which limits its application to gene therapy. Furthermore, only one cell at a time can be microinjected, which limits the technique's efficiency. As a means of introducing dsRNA into cells, electroporation is also relatively impractical, because it is not possible in vivo. Finally, while dsRNA can be introduced into cells using liposome-facilitated transportation or passive uptake, these techniques are slow and inefficient.

It is possible to introduce dsRNA indirectly into cells, by transforming the cells with expression vectors containing DNA coding for dsRNA. See, e.g., U.S. Pat. No. 6,278,039; U.S. Application No. 2002/0006664; WO 99/32619; WO 01/29058; WO 01/68836; and WO 01/96584. Cells transformed with the dsRNA-encoding expression vector will then produce dsRNA in vivo. While this technique is theoretically feasible, there are numerous obstacles that must be overcome (e.g., lack of available expression vectors, low transformation efficiency of expression vectors, and dangers associated with use of expression vectors) before it can be widely used clinically (e.g., in gene therapy) or in industry.

Peptide vectors have also been-used to deliver various macromolecules across plasma membranes. In particular, it is known that an antisense oligonucleotide may be transported into a cell if it is conjugated to a protein/peptide vector. The protein-/peptide-vector-conjugated antisense oligonucleotide will then be taken up by the cell. U.S. Application No. 2002/0009758, for example, discloses a means for transporting antisense nucleotides into cells using a short peptide vector, MPG. The MPG peptide contains a hydrophobic domain derived from the fusion sequence of HIV gp41, and a hydrophilic domain derived from the nuclear localization sequence of SV40 T-antigen. It has been demonstrated that several molecules of the MPG peptide coat the antisense oligonucleotide; once coated, the oligonucleotide can be delivered into cultured mammalian cells in less than 1 hour, with relatively high efficiency (90%). Furthermore, it has been shown that interaction with MPG strongly increases both the oligonucleotide's stability to nucleases, and its ability to cross the plasma membrane.

Similarly, U.S. Pat. No. 6,287,792 discloses a method for delivering antisense oligonucleotides to cells, by first linking the oligonucleotides to biotin. The biotinylated antisense oligonucleotides bind to avidin/avidin-fusion-protein, which acts as a transportation vector to assist the antisense oligonucleotides in crossing cell membranes. U.S. Pat. No. 6,025,140 also discloses the use of vector peptides to deliver antisense molecules across plasma membranes, and specifically discloses the use of penetratin and transportan to transport nucleic acids across cell membranes.

The so-called “cell-penetrating peptides” described above offer certain advantages for protocols involving the translocation of macromolecules into cells, including non-traumatic internalization, limited endosomal degradation, high translocation efficiencies at low concentrations, and delivery to a wide variety of cell types. However, none of the above-noted references discloses the use of vector peptides to transport double-stranded ribonucleic acids across cell membranes for the purpose of achieving RNA interference.

Although there are various methods available for directly and indirectly introducing dsRNA into cells, either in vivo or in culture, it is clear that these methods are generally inefficient, and have practical limitations. Furthermore, while RNAi has been shown to function in cultured neurons and other cell systems, artificial induction of RNAi in cultured cells remains difficult. In particular, the study of protein function in neurons has been hindered by the lack of highly-efficient, minimally-deleterious methods of delivering RNAi compounds to primary mammalian neurons. Based upon the foregoing, then, there exists a need to develop tools, methods, and systems to facilitate, and improve the efficiency of, the introduction of dsRNA into neurons and other cells, for the purpose of achieving RNAi.

### SUMMARY OF THE INVENTION

The inventors have made the surprising discovery that application of synthetic dsRNA (e.g., siRNA) linked to a vector peptide (e.g., penetratin1) results in a fast, nearly-complete uptake of the dsRNA (>95% in 2 h) in cultured cells (e.g., primary mammalian neuronal cultures), and leads to specific knockdown of the targeted protein within hours—without the toxicity associated with current methods. Interestingly, the inventors have found that protein knockdown (evident 6 h after treatment) precedes the decrease in targeted message (evident at 24 h after treatment). Early protein effects are dose-dependent, while mRNA knockdown is not, suggesting that an early, mRNA-like translational repression is one of the earliest effects of siRNAs. The inventors have demonstrated the specificity and efficacy of targeting of endogenous protein and message by biochemical, molecular-genetic, and functional assays. In contrast to current methods, the inventors' technique permits study of protein function across entire culture populations, and produces minimal disturbance of complex culture systems.

Accordingly, the present invention provides a gene-silencing system that includes a ribonucleic acid (RNA) molecule linked to a cell-penetrating peptide, wherein the system is capable of silencing expression of a gene in a cell. Also provided are a pharmaceutical composition that includes the system, a cell that includes the system, a kit that includes the system, use of the system in a method of studying protein function in one or more cells, and use of the system in a method of studying interactions between neurons in culture, and use of the system in a method of studying the function of mRNA.

The present invention further provides a gene-silencing system that includes: (a) a small interfering RNA (siRNA) molecule comprising a duplex region of at least 19 nucleotides, wherein at least one strand of the duplex region is homologous to a portion of mRNA transcribed from a gene, and wherein a strand of the siRNA molecule is modified at the 5′ end for linkage with a cell-penetrating peptide; (b) a cell-penetrating peptide selected from the group consisting of penetratin, transportan, pIsl, TAT, pVEC, MTS, and MAP; and (c) a covalent bond linking the siRNA molecule to the cell-penetrating peptide; wherein the system is capable of silencing expression of the gene in a cell. Also provided are a pharmaceutical composition that includes the system, a cell that includes the system, a kit that includes the system, use of the system in a method of studying protein function in one or more cells, and use of the system in a method of studying interactions between neurons in culture.

Additionally, the present invention provides a culture of primary cells, wherein the cells have been contacted with at least one gene-silencing system that includes a ribonucleic acid (RNA) molecule linked to a cell-penetrating peptide, and wherein the at least one system is capable of silencing expression of a gene in the cells.

The present invention further provides a system for use in genetic screening, wherein the system includes a plurality of RNA molecules, and wherein each RNA molecule is linked to a cell-penetrating peptide.

The present invention still further provides a method for silencing expression of a gene in a cell, by contacting a cell with a gene-silencing system, wherein the system includes a ribonucleic acid (RNA) molecule linked to a cell-penetrating peptide, and wherein the system is capable of silencing expression of a gene in the cell. Also provided is a method for silencing expression of a gene in a cell, by contacting a cell with an amount of a gene-silencing system effective to silence expression of a gene in the cell, wherein the gene-silencing system includes: (a) a small interfering RNA (siRNA) molecule comprising a duplex region of at least 19 nucleotides, wherein at least one strand of the duplex region is homologous to a portion of mRNA transcribed from the gene, and wherein a strand of the siRNA molecule is modified at the 5′ end for linkage with a cell-penetrating peptide; (b) a cell-penetrating peptide selected from the group consisting of penetratin, transportan, pIsl, TAT, pVEC, MTS, and MAP; and (c) a covalent bond linking the siRNA molecule to the cell-penetrating peptide; wherein the system is capable of silencing expression of the gene in the cell.

In addition, the present invention provides a method for performing genetic screening, by: (a) providing a collection of cells; (b) contacting the cells with a system that includes a plurality of RNA molecules, wherein each RNA molecule is linked to a cell-penetrating peptide; (c) determining whether one or more of the RNA molecules cause one or more detectable changes in phenotype of the cells; and (d) identifying one or more genes or proteins responsible for the one or more changes in phenotype of the cells.

The present invention further provides a method for determining the function of a gene in a cell, by: (a) providing a gene-silencing system that includes a ribonucleic acid (RNA) molecule linked to a cell-penetrating peptide, wherein the system is capable of silencing expression of a gene in a cell; (b) contacting a cell with the gene-silencing system, such that expression of a gene is silenced in the cell; (c) assessing the phenotype of the cell resulting from step (b); and (d) comparing the phenotype of the cell in step (c) to that of an appropriate control cell, thereby determining the function of the gene in the cell. Also provided is a method for determining the function of a gene in a cell, by: (a) providing a gene-silencing system that includes: (i) a small interfering RNA (siRNA) molecule comprising a duplex region of at least 19 nucleotides, wherein at least one strand of the duplex region is homologous to a portion of mRNA transcribed from a gene, and wherein a strand of the siRNA molecule is modified at the 5′ end for linkage with a cell-penetrating peptide; (ii) a cell-penetrating peptide selected from the group consisting of penetratin, transportan, pIsl, TAT, pVEC, MTS and MAP; and (iii) a covalent bond linking the siRNA molecule to the cell-penetrating peptide; wherein the system is capable of silencing expression of the gene in the cell; (b) contacting the cell with the gene-silencing system, such that expression of the gene is silenced in the cell; (c) assessing the phenotype of the cell resulting from step (b); and (d) comparing the phenotype of the cell in step (c) to that of an appropriate control cell, thereby determining the function of the gene in the cell.

Additional aspects of the present invention will be apparent in view of the description which follows.

### BRIEF DESCRIPTION OF THE FIGURES

FIG. 1 illustrates that vector-linked small interference RNA (siRNA) is taken up rapidly by neurons. Sympathetic neurons were isolated from newborn mice, and grown on coverglass chamber slides for 5 days. Cultures were then treated with 80 nM V-Casp8-FITC-siRNA. Cells were examined within 10 min by confocal microscopy, to detect uptake of FITC-labelled siRNA.

FIG. 2 shows that vector-linked siRNA remains in the neurons for at least 2 days. Hippocampal neurons were isolated from embryonic day 18 (E18) embryos, and grown on coverglass chamber slides for 5 days. Cultures were then treated with 80 nM V-Casp8-FITC-siRNA. Cells were examined 2 days after treatment, by confocal microscopy, to detect the presence of FITC-labelled siRNA.

FIGS. 3A and 3B illustrate that vector-linked siRNA targeted to caspase-8 inhibits expression of caspase-8 in sympathetic neurons. Sympathetic neurons were isolated from newborn mice, and grown on coverglass chamber slides for 5 days. Cultures were then treated with 80 nM V-Casp8-siRNA for one day, fixed and double-labelled with anti-caspase-8 (green) and Hoechst nuclear stain (blue), and examined with fluorescence microscopy. Caspase-8 activity can be seen in the control culture. Anti-caspase-8 activity is depicted by the arrows (FIG. 3A). No caspase-8 activity is seen in the culture treated with V-Casp8-siRNA; only nuclear staining is seen (FIG. 3B).

FIGS. 4A and 4B show that vector-linked siRNA targeted to caspase-9 inhibits expression of caspase-9 in sympathetic neurons. Sympathetic neurons were isolated from newborn mice, and grown on coverglass chamber slides for 5 days. Cultures were then treated with 40 nM V-Casp9-siRNA for one day, fixed and double-labelled with anti-caspase-9 (green) and Hoechst nuclear stain (blue), and examined with fluorescence microscopy. Caspase-9 activity can be seen in the control culture. Anti-caspase-9 activity is depicted by the arrows (FIG. 4A). No caspase-9 activity is seen in the culture treated with V-Casp9-siRNA; only nuclear staining is seen (FIG. 4B).

FIG. 5 illustrates that vector-linked siRNA targeted to Cu—Zn superoxide-dismutase-1 (SOD1) inhibits SOD1-specific activity. Hippocampal neurons were isolated from E18 embryos, and grown in culture for 5 days. Cells were then treated with various concentrations of V-SOD1i (siRNA targeted to SOD1). After 4 h, cells were harvested and assayed for SOD activity.

FIG. 6 shows that vector-linked siRNA targeted to SOD1 is more effective than vector-linked antisense oligonucleotide. Hippocampal neurons were isolated from E18 embryos, and grown in culture for 5 days. Cells were then treated with various concentrations of either V-SOD1i (siRNA targeted to SOD1) or V-ASOD1 (antisense oligonucleotide targeted to SOD1). After one day of treatment, relative neuronal survival was determined.

FIG. 7 shows that the vector can be linked to the sense or antisense strand of siRNA. Hippocampal neurons were isolated from E18 embryos, and grown in culture for 5 days. Cells were then treated with SOD1-siRNA linked to the sense or antisense strand. After one day of treatment, relative neuronal survival was determined.

FIGS. 8A-8C illustrate that vector-linked siRNAs (V-siRNAs) are taken up with high efficiency by neurons, and are non-toxic. (A) Scheme for linking vector (penetratin1) to siRNA. (B) Uptake of V-siRNA. Hippocampal neurons were treated with fluorescein-labelled siRNA (siRNA-FITC) or vector-linked siRNA-FITC (V-siRNA-FITC). DRAQ5 was added to visualize nuclei. Live cells were imaged every 15 min; images shown were taken at 2 h. Every cell with cytoplasm in the plane of focus showed uptake of V-siRNA-FITC. Cells not fully in the plane of focus showed smaller, and sometimes more intense, parts of nuclei. (C) Phase micrographs of hippocampal cultures after siRNA delivery. Hippocampal cultures were treated with Lipofectamine 2000 and siRNA for Casp3 (Transfected), or with V-Casp3i (V-siRNA). Cells were imaged at 0, 6, and 24 h. The three control wells were counted at 0 h, and the remaining cultures were counted at 24 and 48 h (n=4 wells per treatment and time point). Survival is reported at 24 h, relative to the number of cells at time 0. Treatment of hippocampal neurons with V-siRNA caused minimal morbidity/mortality of cultures. green=siRNA-FITC/V-siRNA-FITC; red=DRAQ5

FIGS. 9A-9J demonstrate that V-siRNA specifically suppresses endogenous protein expression. Neuronal cultures (A-D: sympathetic, E-J: hippocampal) were treated with the indicated V-siRNA (80 nM), and fixed at 6 h. (A) V-SOD1i-1; (C) V-SOD1i-2; (E) V-Casp3i; (G) V-Casp8i; (I) V-Casp9i. Cultures were immunostained for the target protein (green) and a non-targeted protein (red). antibodies=anti-SOD1 and anti-caspase-3 (A-D); anti-caspase-3 and anti-caspase-9 (E, F, I, J); anti-caspase-8 and anti-caspase-9 (G, H)

FIGS. 10A-10G show that translational suppression precedes mRNA destruction. (A) SOD1 enzyme activity. Hippocampal cultures were treated with the indicated concentrations of vector-linked siRNA to SOD1 (V-SOD1i-1), for 6 h, and assayed for SOD1 enzyme activity. (B-C) Western blots for SOD1 and caspase-3. Hippocampal cultures were treated with the indicated concentrations of V-siRNA, for 6 h, and analyzed by Western blotting for targeted protein. Blots were stripped and re-probed for MIAP2 or actin, to control for loading. SOD1 and caspase-3 levels were normalized to MIAP2 or actin, and expressed as % of control, as determined by densitometry. (D) V-SOD1i induces apoptosis of hippocampal neurons more effectively than antisense oligonucleotides. Hippocampal cultures were treated with V-SOD1i-1/2, antisense targeting SOD1 (V-ASOD1), blunt-ended V-SOD1i-1 (V-SOD1bl), or unrelated vector-linked siRNA (V-M2i). Survival was assessed after one day, by counting nuclei in cell lysates (n=3). A representative experiment is illustrated; similar results were obtained 6 different times. Survival is reported relative to untreated cultures, and is given as mean±SEM. (E) V-Casp3i protects hippocampal neurons from hydroxynonenal-induced death. Hippocampal cultures were treated with 10 μM hydroxynonenal (HNE), and with V-Casp3i, antisense targeting caspase-3 (V-Acasp3), or blunt-ended V-Casp3i (V-Casp3bl). Survival was assessed after one day, by counting nuclei in cell lysates (n=3). A representative experiment is illustrated; similar results were obtained 4 different times. Survival is reported relative to untreated cultures, and is given as mean±SEM. (F-G) Relative expression of SOD1 and caspase-3 mRNA. Hippocampal cultures were treated with the indicated constructs, and RNA was harvested after 6 and 24 h. cDNA was analyzed by real-time RT-PCR, using serial dilutions. Each sample was analyzed 3 times. Results were normalized to alpha-tubulin mRNA levels, and are given as the mean±SEM (n=3). Similar results were obtained when normalizing against caspase-2 mRNA levels.

### DETAILED DESCRIPTION OF THE INVENTION

RNAi was discovered by Guo and Kemphues in 1995, when they reported that both the sense and antisense strands of test oligonucleotides disrupted the expression of par-1 in Caenorhabditis elegans, following injection into a cell (Guo et al., Par-1, a gene required for establishing polarity in C. elegans embryos, encodes a putative Ser/Thr kinase that is asymmetrically distributed. Cell, 81:611-20, 1995). In 1998, Fire et al. clearly proved the existence and efficacy of RNAi by injecting into the gut of C. elegans a dsRNA that had been prepared in vitro (Fire et al., Potent and specific genetic interference by double-stranded RNA in Caenorhabditis elegans. Nature, 391:806-11, 1998). The injection of dsRNA into C. elegans resulted in loss of expression of the homologous target gene, not only throughout the worm, but also in its progeny.

Since then, RNAi has been shown to play an important role in regulation of gene expression during development. For example, endogenously-expressed micro-RNAs (mRNAs) regulate left-right neuronal asymmetry in C. elegans (Johnston & Hobert, A microRNA controlling left/right neuronal asymmetry in Caenorhabditis elegans. Nature, 426(6968):845-9, 2003). mRNAs are also known to be temporally regulated in neurons during mammalian brain development (Krichevsky et al., A microRNA array reveals extensive regulation of microRNAs during brain development. RNA, (10): 1274-81, 2003; Kim et al., Identification of many microRNAs that copurify with polyribosomes in mammalian neurons. Proc. Natl. Acad. Sci. USA, 101(1):360-5, 2004). Many more mRNAs of unknown function have been isolated from ribonucleoprotein complexes in rodent and human neuronal cell lines (Dostie et al., Numerous microRNPs in neuronal cells containing novel microRNAs. RNA, 9(2):180-6, 2003).

Historically, neurons have proved refractory to easy genetic manipulation. They are more resistant to transfection than other cell types, and, because they are post-mitotic, stable mutant cell lines cannot be established to counter these low efficiencies. However, the inventors disclose herein that treatment with vector-peptide-linked siRNAs leads to highly-efficient induction of RNAi in mammalian neurons cultured from the central and peripheral nervous system. Specifically, the inventors show that conjugates of siRNA and penetratin1 (Pen1) can be used to deliver active siRNA rapidly to neurons, with nearly complete uptake and very low toxicity. Pen1 is a 16-amino-acid peptide, corresponding to the 3^{rd} helix of the antennapaedia homeodomain protein, that enters cells in a non-receptor-mediated fashion. In accordance with methods disclosed herein, siRNA is linked to Pen1 by a disulfide bond; this bond is cleaved in the reducing environment of the cytoplasm, thereby freeing the siRNA (Muratovska and Eccles, Conjugate for efficient delivery of short interfering RNA (siRNA) into mammalian cells. FEBS Lett., 558(1-3):63-8, 2004). The inventors have previously used Pen1 to deliver antisense oligonucleotides and peptide cargoes to primary neurons in culture.

The rapid uptake of vector-linked siRNA (V-siRNA), and the use of V-siRNA to study treated cultures that do not need to recover from transfection, permits investigation into the effects of siRNA on targeted endogenous protein and message levels at time points earlier than was previously possible in cultured mammalian cells. Accordingly, the inventors took advantage of their rapid and efficient delivery system to examine the early effects of siRNA on protein and message levels in neurons.

In a Drosophila cell-free model system that recapitulates RNAi in vitro, cleavage of mRNA has been shown to occur as soon as 5 min after introduction of siRNAs; however, to date, all published data on RNAi in neurons have been from at least 24 h post-treatment. The inventors' observations of the time course of protein and message down-regulation in this window suggest novel models of RNAi function in neurons and other cell types. In particular, the inventors disclose herein that, in contrast to results seen in cell-free systems, protein knockdown mediated by perfectly-complementary siRNA does not require, and can precede, target mRNA degradation.

In the inventors' experiments, as set forth herein, the only siRNA to have an effect on message levels (V-SOD1i-1) had low free energy at the 5′ end of the antisense strand, with an A:U bond at 3 out of the first 4 positions, including the critical first position. In contrast, the other siRNAs discussed herein—which had an early effect on protein levels, but did not cleave mRNA at 24 h—had the stronger C:G bonds at the 5′ end of the antisense strand of the siRNA, and a lower fraction of A:U bonds in the first 4 pairs (V-SOD1i-2: GUCC; VCasp3i: CAUG). Previous studies (Schwarz et al., Asymmetry in the assembly of the RNAi enzyme complex. Cell, 115(2): 199-208, 2003 and Khvorova et al., Functional siRNAs and mRNAs exhibit strand bias. Cell, 115(2):209-16, 2003) have suggested that the free-energy requirements that have been observed for different base positions in siRNAs are mostly important for enabling incorporation into the RISC—possibly because they facilitate unwinding of the siRNA by a RISC-associated helicase. These earlier studies have also suggested that the efficacy of mRNA cleavage is dependent on other factors, such as the degree of sequence homology and reduced bond strength around the cleavage site.

The inventors' experiments have also demonstrated an mRNA-like early-protein-knockdown effect in primary neurons that, like RISC incorporation, requires homologous siRNAs with 2-nt 3′ overhangs, but does not result in mRNA cleavage for many hours, if at all: even in the presence of siRNA that has a perfect match to targeted endogenous mRNA. In addition, this mRNA-like effect shows a dose dependence that is markedly different from that which was observed for message cleavage. The mRNA-degradation effect which the inventors observed was saturated at concentrations of 8 nM or less of V-siRNA; contrastingly, the early mRNA-like protein knockdown was not saturated, even at concentrations of 80 nM. The dose dependence of the mRNA-like effect suggests that it may not be acting-catalytically, unlike traditional RNAi involving cleavage of message.

The foregoing observations suggest that, perhaps in neurons, a RISC capable of mRNA-like translational repression is immediately assembled upon introduction of siRNA; however, as the observations further suggest, this complex is not capable of catalytically cleaving mRNA until some unspecified triggering event occurs, or co-factor becomes available, and this latter step does not always occur within 24 h. It is also possible that mRNA-like translational repression may proceed by an independent pathway that requires both an siRNA-like structure and sequence homology to a target, but does not have the same free-energy requirements for unwinding, and cannot mediate the cleavage of the targeted mRNA.

A further, more speculative, explanation is suggested by work in developmentally-arrested Drosophila oocytes, in which RNAi against existing target mRNA does not proceed until the target mRNA becomes translationally active. In this model, the observed fast protein knockdown would, indeed, be a result of mRNA degradation, but only of that fraction of mRNA which is available for translation. If this pool of mRNA represents only a small fraction of the total targeted mRNA, its degradation could go undetected. The observed specific-degradation mRNA levels at 24 h, in the case of the application of V-SOD1i, could be attributable to a release of target mRNA from sequestration, as part of the apoptotic death initiated by the knockdown of SOD1 by V-SOD1i-1.

Questions about the mechanism of action of siRNA are important for the development of new biological and therapeutic tools, of course. However, mRNAs also appear to play an important role in the normal function and development of the nervous system. One intriguing possibility is that mRNA may play a role in genetic diseases. Genetic polymorphisms that are normally suppressed by mRNAs may be expressed when the suppressor mRNA is absent, thereby leading to expression of mutant protein and development of disease. It is expected that the techniques described herein will be of use to neuroscientists seeking to answer these and other important questions. Indeed, the combination of new, mechanism-based design rules for siRNAs (Schwarz et al., Asymmetry in the assembly of the RNAi enzyme complex. Cell, 115(2):199-208, 2003 and Khvorova et al., Functional siRNAs and mRNAs exhibit strand bias. Cell, 115(2):209-16, 2003), and the inventors' simple, non-toxic method of inducing RNAi in neurons, opens up the possibility of creating large public libraries of siRNAs for use in genetic screens.

Recent work using gene-expression profiling has shown that siRNAs may have significant homology-related off-target activity (Jackson et al., Expression profiling reveals off-target gene regulation by RNAi. Nat. Biotechnol., 21(6): 6357, 2003). This work suggests that siRNA candidates for experimental and therapeutic use must be carefully screened in the specific cell types against which they will be used, and that experimental results should be validated using multiple siRNAs with differing sequences. The high efficiency and simplicity of the inventors' technique also makes such screens in neurons tractable.

Additionally, the inventors' technique should be amenable to performing knockdowns of multiple proteins, either simultaneously or in sequence. The high efficiency of vector-linked siRNA (V-siRNA) treatment brings a double benefit: (1) it allows for the use of population-wide assays of small changes in biological activity; and (2) it brings the power of RNAi to the study of systems involving interactions between neurons, such as electrically-signaling neuronal networks, cell-surface protein interactions, and secreted proteins.

In view of the foregoing, the present invention provides a membrane-permeable complex for facilitating the delivery of a double-stranded ribonucleic acid molecule into a cell, as well as various uses of the complex. Specifically, it has been found that a cell-penetrating peptide may be covalently bonded to a double-stranded ribonucleic acid molecule to form a membrane-permeable complex. Advantageously, use of the complex yields an unprecedented and unexpected 100% transfection efficiency of dsRNA into neuronal cells. Such unprecedented uptake efficiency allows for the efficient in vivo delivery of dsRNA into tissues, and, by extension, into entire organisms, thereby expanding the therapeutic possibilities of RNA interference applications.

While the present invention is primarily directed to the delivery of a double-stranded ribonucleic acid molecule into a cell, for the purposes of RNA interference, the membrane-permeable complex described herein may also be used to facilitate the delivery of other non-coding RNAs, such as small temporal RNAs, short hairpin RNAs, small nuclear RNAs, small nucleolar RNAs, and micro-RNAs, which may be used in applications other than RNA interference. It is also to be understood that singular forms “a”, “an”, and “the”, as used herein, include the plurals as well, unless the context clearly dictates otherwise. For example, a reference to “a membrane-permeable complex” includes a plurality of such membrane-permeable complexes and equivalents thereof known to those skilled in the art.

The membrane-permeable complex described herein comprises a double-stranded ribonucleic acid molecule, a cell-penetrating peptide, and a covalent bond linking the double-stranded ribonucleic acid molecule to the cell-penetrating peptide. As used herein, a “double-stranded ribonucleic acid molecule” refers to any RNA molecule, or fragment or segment thereof, containing two strands that form an RNA duplex, notwithstanding the presence of single-stranded overhangs of unpaired nucleotides. As further used herein, a double-stranded ribonucleic acid (dsRNA) molecule includes single-stranded RNA molecules that form functional stem-loop structures (e.g., small temporal RNAs, short hairpin RNAs, and micro-RNAs), and thereby produce the structural equivalent of an RNA duplex with single-strand overhangs. The RNA molecule of the present invention may be isolated, purified, native, or recombinant, and may be modified by the addition, deletion, substitution, and/or alteration of one or more nucleotides, including non-naturally-occurring nucleotides, such as those added at 5′ and/or 3′ ends to increase nuclease resistance.

A double-stranded ribonucleic acid molecule for use in the present invention may be any one of a number of non-coding RNAs (i.e., an RNA which is not mRNA, tRNA, or rRNA), including, preferably, a small interfering RNA. The dsRNA may also comprise a small temporal RNA, a small nuclear RNA, a small nucleolar RNA, a short hairpin RNA, or a micro-RNA having either a double-stranded structure or a stem-loop configuration comprising an RNA duplex with or without single-strand overhangs. Small nuclear RNAs are non-coding RNAs that are involved in the splicing of pre-mRNAs in eukaryotes (Will and Lührmann, Significant advances have been made in elucidating the biogenesis pathway and three-dimensional structure of the UsnRNPs, the binding blocks of the spliceosome. Curr. Opin. Cell Biol., 13:290, 2001); small nucleolar RNAs are non-coding RNAs that direct 2′-O-ribose methylation and pseudouridylation of rRNA and tRNA (Kiss, T., Small nucleolar RNA-guided post-transcriptional modification of cellular RNAs. EMBO J., 20:3617, 2001); and micro-RNAs (mRNAs) are very small non-coding RNAs, of approximately 22 nucleotides in length, that appear to be involved in various aspects of mRNA regulation and degradation.

Two mRNAs that are characterized in some detail are the small temporal RNAs (stRNAs), lin4 and let7. These stRNAs control developmental timing in the nematode worm, Caenorhabditis elegans and repress the translation of their target genes by binding to the 3′ untranslated regions of their mRNAs (Riddihough, G., The other RNA world, Science, 296:1259, 2002; Ruvkun, G., Molecular biology. Glimpses of a tiny RNA world. Science, 294:797, 2001; Grosshans and Slack, Micro-RNAs: small is plentiful. J. Cell. Biol., 156:17, 2002). Also well-known are the short hairpin RNAs (shRNAs), which are patterned from endogenously-encoded triggers of the RNA interference pathway (Paddison et al., Short hairpin RNAs (shRNAs) induce sequence specific silencing in mammalian cells. Genes and Dev., 16(8):948-58, 2002).

The precursors of endogenous mRNAs—pre-mRNAs—contain self-complementary sequences, and form short hairpin RNAs that are cleaved by the enzyme, Dicer, to form an siRNA-like species. The two strands of mRNA are often not perfectly complementary to each other, or to the mRNA which they target. The ability of imperfectly-matched siRNAs to function as mRNAs (Doench et al., siRNAs can function as mRNAs. Genes Dev., 17(4):438-42, 2003; Saxena et al., Small RNAs with imperfect match to endogenous mRNA repress translation. Implications for off-target activity of small inhibitory RNA in mammalian cells. J. Biol. Chem. 278(45):44312-9, 2003), and the ability of endogenous mRNAs to induce mRNA cleavage of perfectly-complementary targets (Zeng et al., MicroRNAs and small interfering RNAs can inhibit mRNA expression by similar mechanisms. Proc. Natl. Acad. Sci. USA, 100(17):9779-84, 2003; Llave et al., Cleavage of Scarecrow-like mRNA targets directed by a class of Arabidopsis mRNA. Science, 297(5589):2053-6, 2002), have recently been demonstrated in human cell lines. The consensus model of the pathway is one in which imperfect matching to the target mRNA leads to translational repression, while perfect matching leads to message degradation (Lai, microRNAs: runts of the genome assert themselves. Curr. Biol. Review, 13(23):R925-36, 2003).

The double-stranded RNA molecule of the present invention may be very large, comprising thousands of nucleotides. Alternatively (and preferably, in the case of RNAi protocols involving mammalian cells), the dsRNA molecule may be small, in the range of 21-25 nucleotides. Accordingly, a “small interfering RNA”, as used herein, refers to a double-stranded RNA duplex of any length, with or without single-strand overhangs, wherein at least one strand, putatively the antisense strand, is homologous to the target mRNA to be degraded. In a preferred embodiment, the siRNA of the present invention comprises a double-stranded RNA duplex of at least 19-nucleotides, and, even more-preferably, comprises a 21-nucleotide sense and a 21-nucleotide antisense strand paired so as to have a 19-nucleotide duplex region and a 2-nucleotide overhang at each of the 5′ and 3′ ends. Even more preferably, the 2-nucleotide 3′ overhang comprises 2′ deoxynucleotides (e.g., TT, for improved nuclease resistance).

In a preferred embodiment of the present invention, at least one strand of the double-stranded ribonucleic acid molecule (i.e., the antisense strand) of the membrane-permeable complex is homologous to a portion of mRNA transcribed from the Cu—Zn superoxide-dismutase-1 (SOD1) gene—preferably, the human SOD1 gene. More preferably, the double-stranded ribonucleic acid is a small interfering RNA targeted to the nucleotide sequence of SEQ ID NO:1 or SEQ ID NO:2 (see Example 1 below). As used herein, “homologous” refers to a nucleotide sequence that has at least 80% sequence identity (preferably, at least 90% sequence identity; more preferably, at least 95% sequence identity; and even more preferably, at least 98% sequence identity) to a portion of mRNA transcribed from the target gene. Specifically, the small interfering RNA must be of sufficient homology to guide the RNA-induced silencing complex (RISC) to the target mRNA for degradation. Limited mutations in siRNA, relative to the target mRNA, reduce, but do not entirely abolish, target mRNA. Accordingly, the most preferred embodiment of the present invention comprises an siRNA having 100% sequence identity with the target mRNA.

In another embodiment of the present invention, at least one strand of the double-stranded ribonucleic acid molecule of the membrane-permeable complex is homologous to a portion of mRNA transcribed from a caspase 8 gene—preferably, the human caspase 8 gene. In a preferred embodiment, the double-stranded ribonucleic acid molecule is a small interfering RNA comprising the nucleotide sequence of SEQ ID NO:3 or SEQ ID NO:4 (see Example 1 below). In yet another embodiment of the present invention, at least one strand of the double-stranded ribonucleic acid molecule of the membrane-permeable complex is homologous to a portion of mRNA transcribed from a caspase 9 gene—preferably, the human caspase 9 gene. In a preferred embodiment, the double-stranded ribonucleic acid molecule is a small interfering RNA comprising the nucleotide sequence of SEQ ID NO:5 (see Example 1 below).

In the practice of the present invention, at least one strand of the double-stranded ribonucleic acid molecule (either the sense or the antisense strand) may be modified for linkage with a cell-penetrating peptide (e.g., with a thiol group), so that the covalent bond links the modified strand to the cell-penetrating peptide. Where the strand is modified with a thiol group, the covalent bond linking the cell-penetrating peptide and the modified strand of the ribonucleic acid molecule can be a disulfide bond, as is the case where the cell-penetrating peptide has a free thiol function (i.e., pyridyl disulfide or a free cysteine residue) for coupling. However, it will be apparent to those skilled in the art that a wide variety of functional groups may be used in the modification of the ribonucleic acid, so that a wide variety of covalent bonds (e.g., ester bonds, carbamate bonds, sulfonate bonds, etc.) may be applicable. Additionally, the membrane-permeable complex of the present invention may further comprise a moiety conferring target-cell specificity to the complex.

In a preferred embodiment of the present invention, it is the 5′ end of at least one strand of the double-stranded ribonucleic acid that is modified for linkage with the cell-penetrating peptide (e.g., with a group having a thiol function, such as a 5′ amino-C6 linker); the 3′ OH end of the strand is left free. Alternatively, where activity of the double-stranded ribonucleic acid molecule is not adversely affected (i.e., there is no significant reduction in degradation of target mRNA), at least one strand of the double-stranded ribonucleic acid may be modified at its 3′ end for linkage with the cell-penetrating peptide, where the covalent bond links the 3′ modified strand to the cell-penetrating peptide (Holen et al., Positional effects of short interfering RNAs targeting the human coagulation trigger tissue factor. Nucleic Acids Res., 30(8):1757-66, 2002).

A label (e.g., an enzyme label, a chemical label, a radioactive label, etc.) may also be affixed to at least one strand of the double-stranded ribonucleic acid molecule. Common enzymatic labels include horseradish peroxidase, biotin/avidin/streptavidin labeling, alkaline phosphatase, and beta-galactosidase. Chemical labels used in the present invention encompass all types of fluorophores and fluorescent agents, including but not limited to, fluorescein and its derivatives, rhodamine, cy3, and cy5; fluorescent proteins, such as phycocyanin or green fluorescent protein; and chemiluminescent labels. By way of non-limiting example, fluorescein may be linked to the ribonucleic acid using the reactive derivative, fluorescein isothiocyanate (FITC). Finally, common radioactive labels include ³H, ¹³¹I, and ⁹⁹Tc. Again, in a preferred embodiment, the label is affixed to the 5′ end of the strand, although the label may be attached at the 3′ end of the strand where such attachment does not significantly affect the activity of the double-stranded ribonucleic acid molecule. Additionally, labels can be attached to the body of the siRNA post-synthesis.

The membrane-permeable complex described herein comprises a cell-penetrating peptide covalently bound to the double-stranded ribonucleic acid molecule. Several features make cell-penetrating peptides unique vehicles for transporting biologically-important molecules into cells. In particular, the activity of cell-penetrating peptides is generally non-cell-type specific. Additionally, cell-penetrating peptides typically function with high efficiency, even at low concentrations; furthermore, the penetration of cell-penetrating peptides through cell membranes is often independent of endocytosis, energy requirements, receptor molecules, and transporter molecules. Thus, cell-penetrating peptides can efficiently deliver large cargo molecules into a wide variety of target cells (Derossi et al., Trojan peptides: the penetratin system for intracellular delivery. Trends Cell Biol., 8(2):84-87, 1998; Dunican et al., Designing cell-permeant phosphopeptides to modulate intracellular signaling pathways. Biopolymers, 60(1):45-60, 2001; Hallbrink et al., Cargo delivery kinetics of cell-penetrating peptides, Biochim. Biophys. Acta, 1515(2): 101-09, 2001; Bolton et al., Cellular uptake and spread of the cell-permeable peptide penetratin in adult rat brain. Eur. J. Neurosci., 12(8):2847-55, 2000; Kilk et al., Cellular internalization of a cargo complex with a novel peptide derived from the third helix of the islet-1 homeodomain. Comparison with the penetratin peptide. Bioconjug. Chem., 12(6):911-16, 2001).

As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIsl, TAT(48-60), pVEC, MTS, and MAP.

In the most preferred embodiment of the present invention, the cell-penetrating peptide of the membrane-permeable complex is penetratin, comprising the peptide sequence RQIKIWFQNRRMKWKK (SEQ ID NO:6), or a conservative variant thereof. As used herein, a “conservative variant” is a peptide having one or more amino acid substitutions, wherein the substitutions do not adversely affect the shape—or, therefore, the biological activity (i.e., transport activity) or membrane toxicity—of the cell-penetrating peptide.

Penetratin is a 16-amino-acid polypeptide derived from the third alpha-helix of the homeodomain of Drosophila antennapedia. Its structure and function have been well studied and characterized: Derossi et al., Trojan peptides: the penetratin system for intracellular delivery. Trends Cell Biol., 8(2):84-87, 1998; Dunican et al., Designing cell-permeant phosphopeptides to modulate intracellular signaling pathways. Biopolymers, 60(1):45-60, 2001; Hallbrink et al., Cargo delivery kinetics of cell-penetrating peptides, Biochim. Biophys. Acta, 1515(2):101-09, 2001; Bolton et al., Cellular uptake and spread of the cell-permeable peptide penetratin in adult rat brain. Eur. J. Neurosci., 12(8):2847-55, 2000; Kilk et al., Cellular internalization of a cargo complex with a novel peptide derived from the third helix of the islet-1 homeodomain. Comparison with the penetratin peptide. Bioconjug. Chem., 12(6):911-16, 2001; Bellet-Amalric et al., Interaction of the third helix of antennapedia homeodomain and a phospholipid monolayer, studied by ellipsometry and PM-IRRAS at the air-water interface. Biochim. Biophys. Acta, 1467(1):131-43, 2000; Fischer et al., Structure-activity relationship of truncated and substituted analogues of the intracellular delivery vector. Penetratin. J. Pept. Res., 55(2): 163-72, 2000; Thoren et al., The antennapedia peptide penetratin translocates across lipid bilayers—the first direct observation. FEBS Lett., 482(3):265-68, 2000.

It has been shown that penetratin efficiently carries avidin, a 63-kDa protein, into human Bowes melanoma cells (Kilk et al., Cellular internalization of a cargo complex with a novel peptide derived from the third helix of the islet-1 homeodomain. Comparison with the penetratin peptide. Bioconjug. Chem., 12(6):911-16, 2001). Additionally, it has been shown that the transportation of penetratin and its cargo is non-endocytotic and energy-independent, and does not depend upon receptor molecules or transporter molecules. Furthermore, it is known that penetratin is able to cross a pure lipid bilayer (Thoren et al., The antennapedia peptide penetratin translocates across lipid bilayers—the first direct observation. FEBS Lett., 482(3):265-68, 2000). This feature enables penetratin to transport its cargo, free from the limitation of cell-surface-receptor/-transporter availability. The delivery vector previously has been shown to enter all cell types (Derossi et al., Trojan peptides: the penetratin system for intracellular delivery. Trends Cell Biol., 8(2):84-87, 1998), and effectively to deliver peptides (Troy et al., The contrasting roles of ICE family proteases and interleukin-1beta in apoptosis induced by trophic factor withdrawal and by copper/zinc superoxide dismutase down-regulation. Proc. Natl. Acad. Sci. USA, 93:5635-40, 1996) or antisense oligonucleotides (Troy et al., Downregulation of Cu/Zn superoxide dismutase leads to cell death via the nitric oxide-peroxynitrite pathway. J. Neurosci., 16:253-61, 1996; Troy et al., Nedd2 is required for apoptosis after trophic factor withdrawal, but not superoxide dismutase (SOD1) downregulation, in sympathetic neurons and PC12 cells. J. Neurosci., 17:1911-18, 1997).

Other cell-penetrating peptides that may be used in accordance with the present invention include transportan, pISl, Tat(48-60), pVEC, MAP, and MTS. Transportan is a 27-amino-acid long peptide containing 12 functional amino acids from the amino terminus of the neuropeptide galanin, and the 14-residue sequence of mastoparan in the carboxyl terminus, connected by a lysine (Pooga et al., Cell penetration by transportan. FASEB J., 12(1):67-77, 1998). It comprises the amino acid sequence GWTLNSAGYLL GKINLKALAALAKKIL (SEQ ID NO:7), or a conservative variant thereof.

pIsl is derived from the third helix of the homeodomain of the rat insulin 1 gene enhancer protein (Magzoub et al., Interaction and structure induction of cell-penetrating peptides in the presence of phospholipid vesicles. Biochim. Biophys. Acta, 1512(1):77-89, 2001; Kilk et al., Cellular internalization of a cargo complex with a novel peptide derived from the third helix of the islet-1 homeodomain. Comparison with the penetratin peptide. Bioconjug. Chem., 12(6):911-16, 2001). pIsl comprises the amino acid sequence PVIRVW FQNKRCKDKK (SEQ ID NO:8), or a conservative variant thereof.

Tat is a transcription activating factor, of 86-102 amino acids, that allows translocation across the plasma membrane of an HIV-infected cell, to transactivate the viral genome (Hallbrink et al., Cargo delivery kinetics of cell-penetrating peptides. Biochim. Biophys. Acta., 1515(2):101-09, 2001; Suzuki et al., Possible existence of common internalization mechanisms among arginine-rich peptides. J. Biol. Chem., 277(4):2437-43, 2002; Futaki et al., Arginine-rich peptides. An abundant source of membrane-permeable peptides having potential as carriers for intracellular protein delivery. J. Biol. Chem., 276(8):5836-40, 2001). A small Tat fragment, extending from residues 48-60, has been determined to be responsible for nuclear import (Vives et al., A truncated HIV-1 tat protein basic domain rapidly translocates through the plasma membrane and accumulates in the cell nucleus. J. Biol. Chem., 272(25):16010-017, 1997); it comprises the amino acid sequence GRKKRRQRRRPPQ (SEQ ID NO:9), or a conservative variant thereof.

pVEC is an 18-amino-acid-long peptide derived from the murine sequence of the cell-adhesion molecule, vascular endothelial cadherin, extending from amino acid 615-632 (Elmquist et al., VE-cadherin-derived cell-penetrating peptide, pVEC, with carrier functions. Exp. Cell Res., 269(2):237-44, 2001). pVEC comprises the amino acid sequence LLIILRRRIRKQAHAH (SEQ ID NO:10), or a conservative variant thereof.

MTSs, or membrane translocating sequences, are those portions of certain peptides which are recognized by the acceptor proteins that are responsible for directing nascent translation products into the appropriate cellular organelles for further processing (Lindgren et al., Cell-penetrating peptides. Trends in Pharmacological Sciences, 21(3):99-103, 2000; Brodsky, J. L., Translocation of proteins across the endoplasmic reticulum membrane. Int. Rev. Cyt., 178:277-328, 1998; Zhao et al., Chemical engineering of cell penetrating antibodies. J. Immunol. Methods, 254(1-2):137-45, 2001). An MTS of particular relevance is MPS peptide, a chimera of the hydrophobic terminal domain of the viral gp41 protein and the nuclear localization signal from simian virus 40 large antigen; it represents one combination of a nuclear localization signal and a membrane translocation sequence that is internalized independent of temperature, and functions as a carrier for oligonucleotides (Lindgren et al., Cell-penetrating peptides. Trends in Pharmacological Sciences, 21(3):99-103, 2000; Morris et al., A new peptide vector for efficient delivery of oligonucleotides into mammalian cells. Nucleic Acids Res., 25:2730-36, 1997). MPS comprises the amino acid sequence GALFLGWLGAAGSTMGAWSQPKKKRKV (SEQ ID NO:11), or a conservative variant thereof.

Model amphipathic peptides, or MAPs, form a group of peptides that have, as their essential features, helical amphipathicity and a length of at least four complete helical turns (Scheller et al., Structural requirements for cellular uptake of alpha-helical amphipathic peptides. J. Peptide Science, 5(4):185-94, 1999; Hallbrink et al., Cargo delivery kinetics of cell-penetrating peptides. Biochim. Biophys. Acta., 1515(2):101-09, 2001). An exemplary MAP comprises the amino acid sequence KLALKLALKALKAALKLA-amide (SEQ ID NO:12), or a conservative variant thereof.

The cell-penetrating peptides and the double-stranded ribonucleic acids described above are covalently bound to form membrane-permeable complexes in accordance with the present invention. A general strategy for conjugation involves preparing the cell-penetrating peptide and double-stranded ribonucleic acid components separately, wherein each is modified or derivatized with appropriate reactive groups to allow for linkage between the two. The modified double-stranded ribonucleic acid is then incubated together with a cell-penetrating peptide that is prepared for linkage, for a sufficient time (and under such appropriate conditions of temperature, pH, molar ratio, etc.) as to generate a covalent bond between the cell-penetrating peptide and the double-stranded ribonucleic acid molecule.

Numerous methods and strategies of conjugation will be readily apparent to one of ordinary skill in the art, as will the conditions required for efficient conjugation. By way of example only, one such strategy for conjugation is described below.

In order to generate a disulfide bond between the double-stranded ribonucleic acid molecule and the cell-penetrating peptide of the present invention, the 3′ or 5′ end of the dsRNA molecule may be modified with a thiol group, and a nitropyridyl leaving group may be manufactured on a cysteine residue of the cell-penetrating peptide. Any suitable bond (e.g., thioester bonds, thioether bonds, carbamate bonds, etc.) may be created, however, according to methods generally and well known in the art. Both the derivatized or modified cell-penetrating peptide, and the modified double-stranded ribonucleic acid, are reconstituted in RNase/DNase sterile water, and then added to each other in amounts appropriate for conjugation (e.g., equimolar amounts). The conjugation mixture is then incubated for 15 min at 65° C., followed by 60 min at 37° C., and then stored at 4° C. Linkage can be checked by running the vector-linked siRNA, and an aliquot that has been reduced with DTT, on a 15% non-denaturing PAGE. siRNA can then be visualized with SyBrGreen.

The present invention further provides a gene-silencing system comprising a ribonucleic acid (RNA) molecule linked to a cell-penetrating peptide, wherein the system is capable of silencing expression of a gene in a cell. As described above, the RNA molecule may be modified for linkage with the cell-penetrating peptide and/or linked to the cell-penetrating peptide by a disulfide bond. The RNA molecule may also be single-stranded or double-stranded. By way of example, the single-stranded RNA molecule (ssRNA) may have a hairpin structure; the ssRNA molecule may also be a micro-RNA (mRNA) or a precursor thereof. The double-stranded RNA (dsRNA) molecule may be a small interfering RNA (siRNA), for example. In one embodiment of the present invention, a strand of the dsRNA molecule is modified at the 5′ end (e.g., modified with a thiol group) for linkage with the cell-penetrating peptide, as described above. The modified 5′ end of the strand of the dsRNA molecule may be linked to the cell-penetrating peptide by a covalent bond (e.g., a disulfide bond), as described above.

In accordance with the present invention, the RNA molecule of the gene-silencing system may specifically bind to mRNA transcribed from a gene of interest. For example, the RNA molecule may be substantially homologous to, or complementary to, a portion of the transcribed mRNA. The gene of interest may be any gene now known or later discovered, and may be obtained from, or located in, any organism, including a mammal (e.g., a human). In one embodiment, the gene is Cu—Zn superoxide dismutase-1 (SOD1) gene, a caspase 3 (Casp3) gene, a caspase 8 (Casp8) gene, or a caspase 9 (Casp9) gene. In another embodiment, the RNA molecule comprises a nucleic acid having one of the following nucleotide sequences: SEQ ID NO:1, SEQ ID NO:2, SEQ ID NO:3, SEQ ID NO:4, SEQ ID NO:5, SEQ ID NO:13, or SEQ ID NO:14, as disclosed herein.

The gene-silencing system of the present invention may comprise any cell-penetrating peptide, including, without limitation, penetratin, transportan, pIsl, TAT, pVEC, MTS, and MAP, as described above. In one preferred embodiment, the cell-penetrating peptide is penetratin. In general, the RNA molecule of the gene-silencing system will be released from the cell-penetrating peptide inside the cell.

In accordance with the present invention, the gene-silencing system described herein may be used to silence expression of a gene in any cell. In certain embodiments, the cell is a post-mitotic cell (e.g., a neuron). The cell may also be a cell of a primary culture. Once inside the cell, the RNA molecule may silence expression of the gene at the protein and/or RNA level. In one embodiment of the present invention, expression of the gene is silenced only at the protein level. In another embodiment, expression of the gene is silenced at both the protein and RNA levels (e.g., silencing at the protein level is followed by silencing at the RNA level). The gene-silencing system of the present invention is particularly desirable in that it has the ability to silence expression of a gene in a cell without RNA-independent cytotoxicity.

The gene-silencing system disclosed herein may further comprise other molecular entities. By way of example, the gene-silencing system may further comprise at least one label (e.g., an enzyme label, a chemical label, or a radioactive label) affixed to the RNA molecule, as described above. In one embodiment, the one or more labels are affixed to the 5′ end of a strand of the RNA molecule. Additionally, the gene-silencing system may further comprise a moiety conferring target-cell specificity to the system.

The present invention also provides a gene-silencing system comprising: (a) a small interfering RNA (siRNA) molecule comprising a duplex region of at least 19 nucleotides, wherein at least one strand of the duplex region is homologous to a portion of mRNA transcribed from a gene, and wherein a strand of the siRNA molecule is modified at the 5′ end for linkage with a cell-penetrating peptide; (b) a cell-penetrating peptide selected from the group consisting of penetratin, transportan, pIsl, TAT, pVEC, MTS, and MAP; and (c) a covalent bond linking the siRNA molecule to the cell-penetrating peptide. This system is capable of silencing expression of a gene in a cell.

The present invention further provides a cell comprising a membrane-permeable complex or gene-silencing system as described herein. In certain embodiments, the cell is a post-mitotic cell (e.g., a neuron). The cell may also be a cell of a primary culture. In one embodiment, the membrane-permeable complex or gene-silencing system silences expression of the gene in the cell. The present invention also provides a kit for silencing expression of a gene in a cell, comprising: (a) a membrane-permeable complex or gene-silencing system, as described herein; and (b) optionally, instructions for using the complex or system.

Additionally, the present invention provides a cell culture comprising primary cells that have been contacted with at least one membrane-permeable complex or gene-silencing system, as described herein, wherein the at least one complex or system is capable of silencing expression of a gene in the cells. For example, the primary cells may be contacted with at least one gene-silencing system comprising a ribonucleic acid (RNA) molecule linked to a cell-penetrating peptide. The primary cells of the cell culture may be cells of any organism (e.g., a mammal), and may be any type of cells, including, without limitation, neurons (e.g., hippocampal neurons). In one embodiment of the present invention, the at least one membrane-permeable complex or gene-silencing system silences expression of the gene in one or more cells in the culture. In another embodiment, the RNA molecule of the membrane-permeable complex or gene-silencing system is labelled with a fluorescent dye (e.g., fluorescein).

The present invention further provides a system for use in genetic screening. The system comprises a plurality of RNA molecules, wherein each RNA molecule is linked to a cell-penetrating peptide. In one embodiment of the present invention, at least one of the RNA molecules, when contacted with a cell (e.g., a neuron), has an effect on at least one cellular event in the cell. The at least one cellular event in the cell may result in at least one detectable change in phenotype of the cell. The system of the present invention may further comprise at least one label affixed to at least one of the RNA molecules.

The present invention further provides various methods of using the membrane-permeable complexes and gene-silencing systems described herein. For example, a method for facilitating delivery of a double-stranded ribonucleic acid molecule into a cell is disclosed. In accordance with this method, a membrane-permeable complex, comprising a double-stranded ribonucleic acid molecule, a cell-penetrating peptide, and a covalent bond linking the double-stranded ribonucleic acid molecule to the cell-penetrating peptide, is contacted with the cell, thereby resulting in delivery of the double-stranded ribonucleic acid molecule into the cell. The membrane-permeable complex is contacted with the cell for such a period of time—and under such conditions of concentration, temperature, pH, etc.—as to result in delivery of the complex into the cell. Specific protocols using the membrane-permeable complex of the present invention will vary according to a number of factors (such as cell type, passage number, cell-penetrating peptide used, etc.), but will be readily apparent to one of ordinary skill in the art.

In one preferred embodiment of the present invention, at least one strand of the double-stranded ribonucleic acid molecule is modified at its 5′ end for linkage with the cell-penetrating peptide, and the covalent bond links the 5′ modified strand to the cell-penetrating peptide. The 5′ end may be modified with a group having a thiol function, and the covalent bond linking the modified 5′ end with the cell-penetrating peptide may be a disulfide bond, as would be the case where the cell-penetrating peptide has a free thiol group (or a group of corresponding function) for attachment. Alternatively, where function of the double-stranded ribonucleic acid molecule is not adversely affected by such modification, at least one strand of the double-stranded ribonucleic acid molecule may be modified at its 3′ end for linkage with the cell-penetrating peptide, where the covalent bond links the 3′ modified strand to the cell-penetrating peptide. In another preferred embodiment, the double-stranded ribonucleic acid molecule is a small interfering RNA, although other embodiments of the disclosed method contemplate the use of other non-coding RNAs, including small temporal RNAs, small nuclear RNAs, small nucleolar RNAs, short hairpin RNAs, and micro-RNAs.

Where the membrane-permeable complex of the present invention is delivered to a cell for the purpose of inhibiting expression of a target gene within the cell (i.e., for RNA interference), the double-stranded ribonucleic acid molecule that is delivered as part of the membrane-permeable complex is preferably a small interfering RNA. Furthermore, preferably at least one strand of the small interfering RNA is homologous to a portion of mRNA transcribed from the target gene. In a preferred embodiment, the siRNA strand is at least 85% homologous to a portion of mRNA transcribed from the target gene. Preferably, the siRNA strand is 90% homologous to a portion of mRNA transcribed from the target gene; more preferably, the siRNA strand is 95% homologous to a portion of mRNA transcribed from the target gene; even more preferably, the siRNA strand is 98% homologous to a portion of mRNA transcribed from the target gene. In the most preferred embodiment, at least one strand of the siRNA is 100% homologous to a portion of mRNA transcribed from the target gene.

The target gene of the present invention may be a gene that is endogenous to the cell, as in the case of a regulatory gene or a gene coding for a native protein; alternatively, the target gene may be heterologous or exogenous in relation to the cell, as in the case of a viral or bacterial gene, transposon, or transgene. In either case, uninhibited expression of the target gene may result in a disease or a condition. The cell is contacted with the membrane-permeable complex so that the complex is delivered into the cell in an amount sufficient to inhibit expression of the target gene. Such delivery may be accomplished in vitro, in vivo, or ex vivo.

The cell receiving the membrane-permeable complex of the present invention may be isolated, within a tissue, or within an organism. It may be an animal cell, a plant cell, a fungal cell, a protozoan, or a bacterium. An animal cell may be derived from vertebrates or invertebrates. However, in a preferred embodiment of the invention, the cell is derived from a mammal, such as a rodent or a primate; more preferably, the cell is derived from a human. The cell may be any type of cell, including an epithelial cell, an endothelial cell, a muscle cell, and a nerve cell. Representative cell types include, but are not limited to, myoblasts, fibroblasts, astrocytes, neurons, oligodendrocytes, macrophages, myotubes, lymphocytes, NIH3T3 cells, PC12 cells, and neuroblastoma cells. In a preferred embodiment, the cell is a neuron.

Similarly, the present invention provides a method for silencing expression of a gene in a cell, comprising contacting a cell with a gene-silencing system, as described herein. In one embodiment, the gene-silencing system is capable of silencing expression of a gene in the cell, and comprises a ribonucleic acid (RNA) molecule linked to a cell-penetrating peptide. In another embodiment, the gene-silencing system is capable of silencing expression of the gene in the cell, and comprises: (a) a small interfering RNA (siRNA) molecule comprising a duplex region of at least 19 nucleotides, wherein at least one strand of the duplex region is homologous to a portion of mRNA transcribed from the gene, and wherein a strand of the siRNA molecule is modified at the 5′ end for linkage with a cell-penetrating peptide; (b) a cell-penetrating peptide selected from the group consisting of penetratin, transportan, pIsl, TAT, pVEC, MTS, and MAP; and (c) a covalent bond linking the siRNA molecule to the cell-penetrating peptide.

In accordance with methods described herein, expression of a gene may be silenced in a cell at the protein and/or RNA level. In one embodiment of the present invention, expression of the gene is silenced only at the protein level. In another embodiment, expression of the gene is silenced at both the protein and RNA levels (e.g., silencing at the protein level is followed by silencing at the RNA level). In general, the RNA molecule of the membrane-permeable complex or gene-silencing system will be released from the cell-penetrating peptide inside the cell. The cell may be any of those described above. Preferably, the cell is a neuron.

In the methods of the present invention, a cell or tissue may be contacted with a membrane-permeable complex or gene-silencing system ex vivo. Alternatively, a cell or tissue may be contacted with a membrane-permeable complex or gene-silencing system in vivo in a subject. Where the membrane-permeable complex or gene-silencing system of the present invention is delivered in vivo to a living organism, administration may be achieved by any procedure known in the art, including without limitation, intradermal, parenteral, rectal, in situ (e.g., delivery using a catheter), topical, and/or transdermal administration to the subject.

To facilitate delivery to a cell, tissue, or subject, the membrane-permeable complex or gene-silencing system of the present invention may, in various compositions, be formulated with a pharmaceutically-acceptable carrier, excipient, or diluent. The term “pharmaceutically-acceptable”, as used herein, means that the carrier, excipient, or diluent of choice does not adversely affect either the biological activity of the membrane-permeable complex/gene-silencing system or the biological activity of the recipient of the composition. Suitable pharmaceutical carriers, excipients, and/or diluents for use in the present invention include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water. Specific formulations of compounds for therapeutic treatment are discussed in Hoover, J. E., Remington's Pharmaceutical Sciences (Easton, Pa.: Mack Publishing Co., 1975) and Liberman and Lachman, eds., Pharmaceutical Dosage Forms (New York, N.Y.: Marcel Decker Publishers, 1980).

For oral administration, the composition of the present invention may be presented as capsules or tablets, powders, granules, or a suspension. The composition may be further presented in convenient unit-dosage form, and may be prepared using a controlled-release formulation, buffering agents, and/or enteric coatings.

For parenteral administration (i.e., subcutaneous, intravenous, or intramuscular administration), the membrane-permeable complex or gene-silencing system may be dissolved or suspended in a sterile aqueous or non-aqueous isotonic solution, containing one or more of the carriers, excipients, or diluents noted above. Such formulations may be prepared by dissolving a composition containing the membrane-permeable complex or gene-silencing system in sterile water containing physiologically-compatible substances (such as sodium chloride, glycine, and the like), and having a buffered pH compatible with physiological conditions, so as to produce an aqueous solution. Alternatively, a composition containing the membrane-permeable complex or gene-silencing system may be dissolved in non-aqueous isotonic solutions of polyethylene glycol, propylene glycol, ethanol, corn oil, cottonseed oil, peanut oil, etc.

The membrane-permeable complex or gene-silencing system of the present invention may be administered rectally by formulation with any suitable carrier that is solid at room temperature, but dissolves at body temperature. Such carriers include cocoa butter, synthetic mono-, di-, or tri-glycerides, fatty acids, polyethylene glycols, glycerinated gelatin, hydrogenated vegetable oils, and the like.

Intradermal administration of the membrane-permeable complex or gene-silencing system (i.e., administration via an injectable preparation) may be accomplished by suspending or dissolving the membrane-permeable complex or gene-silencing system in a non-toxic, parenterally-acceptable diluent or solvent (e.g., as a solution in 1,3-butanediol, water, Ringer's solution, and isotonic sodium chloride solution). Occasionally, sterile-fixed oils or fatty acids are employed as a solvent or suspending medium.

For transdermal or topical administration, the membrane-permeable complex or gene-silencing system may be combined with compounds that act to increase the permeability of the skin and allow passage of the membrane-permeable complex or gene-silencing system into the bloodstream. Such enhancers include propylene glycol, polyethylene glycol, isopropanol, ethanol, oleic acid, N-methylpyrrolidone, and the like. Delivery of such compositions may be accomplished via transdermal patch or iontophoresis device.

In accordance with the method of the present invention, the quantity of the membrane-permeable complex or gene-silencing system that is administered to a cell, tissue, or subject should be an amount that is effective to inhibit expression of the target gene within the tissue or subject. This amount is readily determined by the practitioner skilled in the art. The specific dosage will depend upon a number of factors, including the type of RNA used (e.g., siRNA), the target gene to be inhibited, and the cell type having target-gene expression. Quantities will be adjusted for the body weight of the subject, and the particular disease or condition being targeted.

Additionally, the present invention discloses a method for determining the function of a target gene in a cell. First, a membrane-permeable complex for inhibiting expression of the target gene is contacted with the cell, wherein the membrane-permeable complex comprises: (i) a double-stranded ribonucleic acid molecule, wherein at least one strand of the molecule has a nucleotide sequence that is homologous to a portion of mRNA transcribed from the target gene; (ii) a cell-penetrating peptide; and (iii) a covalent bond linking the double-stranded ribonucleic acid molecule to the cell-penetrating peptide. Once the complex is delivered into the cell, in an amount sufficient to inhibit expression of the target gene, the phenotype of the contacted cell is compared to that of an appropriate control cell, thereby allowing for the determination of information regarding the function of the target gene in the cell.

The present invention further provides a method for determining the function of a gene in a cell, comprising the steps of: (a) providing a gene-silencing system comprising a ribonucleic acid (RNA) molecule linked to a cell-penetrating peptide, wherein the system is capable of silencing expression of a gene in a cell; (b) contacting a cell with the gene-silencing system, such that expression of the gene is silenced in the cell; (c) assessing the phenotype of the cell resulting from step (b); and (d) comparing the phenotype of the cell in step (c) to that of an appropriate control cell, thereby determining the function of the gene in the cell. Similarly, the present invention provides a method for determining the function of a gene in a cell, comprising the steps of: (a) providing a gene-silencing system which is capable of silencing expression of the gene in the cell, comprising: (i) a small interfering RNA (siRNA) molecule comprising a duplex region of at least 19 nucleotides, wherein at least one strand of the duplex region is homologous to a portion of mRNA transcribed from a gene, and wherein a strand of the siRNA molecule is modified at the 5′ end for linkage with a cell-penetrating peptide; (ii) a cell-penetrating peptide selected from the group consisting of penetratin, transportan, pIsl, TAT, pVEC, MTS, and MAP; and (iii) a covalent bond linking the siRNA molecule to the cell-penetrating peptide; (b) contacting the cell with the gene-silencing system, such that expression of the gene is silenced in the cell; (c) assessing the phenotype of the cell resulting from step (b); and (d) comparing the phenotype of the cell in step (c) to that of an appropriate control cell, thereby determining the function of the gene in the cell.

The present invention also provides a method for performing genetic screening, comprising the steps of: (a) providing a collection of cells; (b) contacting the cells with a system comprising a plurality of RNA molecules, wherein each RNA molecule is linked to a cell-penetrating peptide; (c) determining if one or more of the RNA molecules cause one or more detectable changes in phenotype of the cells; and (d) identifying one or more genes or proteins responsible for the one or more changes in phenotype of the cells. Also provided is use of a gene-silencing system, as disclosed herein, in a method of studying protein function in one or more cells and/or in a method of studying interactions between neurons in culture (e.g., electrical signaling in neural networks, cell-surface-protein expression, secreted-protein expression, etc.).

The present invention is described in the following Examples, which are set forth to aid in the understanding of the invention, and should not be construed to limit in any way the scope of the invention as defined in the claims which follow thereafter.

### EXAMPLES

### Example 1

### siRNA Synthesis

Targets for siRNA were designed for various mRNAs. A general strategy for designing siRNA targets begins with an AUG stop codon; the length of the desired cDNA is then scanned for AA dinucleotide sequences. The 3′ 19 nucleotides adjacent to the AA sequences are recorded as potential siRNA target sites. The potential target sites can then be compared to the appropriate genome databases, so that any target sequences that have significant homology to non-target genes can be discarded. Multiple target sequences along the length of the gene should be located, so that target sequences are derived from the 3′, 5′, and medial portions of the mRNA. Negative-control siRNAs can be generated using the same nucleotide composition as the subject siRNA, provided it has been scrambled and checked to ensure that it lacks sequence homology to any genes of the cells being transfected (Elbashir et al., Duplexes of 21-nucleotide RNAs mediate RNA interference in cultured mammalian cells, Nature, 411:494-98, 2001; Ambion siRNA Design Protocol, at www.ambion.com).

In the present case, generated target sequences were 21 bases long (beginning with AA), and modified to have a thiol group at the 5′ C6 carbon on one strand. Custom siRNAs were generated on order from Dharmacon Research, Inc. (Lafayette, Colo.). Other sources for custom siRNA preparation include Xeragon Oligonucleotides (Huntsville, Ala.) and Ambion (Austin, Tex.). Alternatively, siRNAs can be chemically synthesized using ribonucleoside phosphoramidites and a DNA/RNA synthesizer. Sequences (sense strands) to which the siRNAs were designed are set forth below:
SOD1 (5′ thiol on sense): AAU CCU CAC UCU AAG AAA CAU (SEQ ID NO:1) (GenBank Accession No. M25157; initiation at base 59; target bases 135-155);
SOD1 (5′ thiol on antisense): AAC CAG UGG UGG UGU CAG GAC (SEQ ID NO:2) (GenBank Accession No. NM_{—}017050; initiation at base 94; target bases 289-309);
Casp8 (5′ thiol on antisense; 5′ FITC on sense): AAG CAC AGA GAG AAG AAU GAG (SEQ ID NO:3) (GenBank Accession No. BC006737; initiation at base 336; target bases 878-898);
Casp8 (5′ thiol on antisense): AAG AAG CAG GAG ACC AUC GAG (SEQ ID NO:4) (GenBank Accession No. BC006737; initiation at base 336; target bases 432-452); and
Casp9 (5′ thiol on antisense): AAG GCA CCC UGG CUU CAC UCU (SEQ ID NO:5) (GenBank Accession No. NM_{—}015733; initiation at base 1; target bases 245-265).

### Example 2

### Linking Penetratin1 to siRNA

Penetratin1 (mw 2503.93) (QBiogene, Inc., Carlsbad, Calif.) was reconstituted to 2 mg/ml in RNase/DNase sterile water (0.8 mM). siRNA (double-stranded, annealed, and synthesized with a 5′ thiol group on the sense or antisense strand) was reconstituted to 88 μM in RNase-/DNase-free sterile water. To link the penetratin1 to the siRNA, 25 μl of penetratin1 were added to 225 μl of the diluted oligo, for a total volume of 250 μl. This mixture was incubated for 15 min at 65° C., followed by 60 min at 37° C., and then stored at 4° C. Where only small amounts of the mixture are required, these may be aliquoted and stored at −80° C. Linkage can be checked by running the vector-linked siRNA, and an aliquot that has been reduced with DTT, on a 15% non-denaturing PAGE. siRNA can be visualized with SyBrGreen (Molecular Probes, Eugene, Oreg.).

### Example 3

### Cell Cultures

Cell cultures used in Examples 5-8 were prepared as follows. Sympathetic neuron cultures were prepared from 1-day-old wild-type and caspase-2^{-/-} mouse pups (Bergeron et al., Defects in regulation of apoptosis in caspase-2-deficient mice, Genes Dev., 12:1304-14, 1998), as previously described (Troy et al., Caspase-2 mediates neuronal cell death induced by beta-amyloid, J. Neurosci., 20:1386-92, 2000). Cultures were grown in 24-well collagen-coated dishes for survival experiments, and in 6-well collagen-coated dishes for RNA and protein extraction in RPMI 1640 medium (Omega Scientific, Tarzana, Calif.; ATCC, Manassas, Va.) plus 10% horse serum with mouse NGF (100 ng/ml). One day following plating, uridine and 5-fluorodeoxyuridine (10 μM each) were added to the cultures, and left for 3 days to eliminate non-neuronal cells. (Less than 1% non-neuronal cells remained after 3 days.)

Hippocampi were dissected from embryonic day 18 (E18) rat fetuses, dissociated by trituration in serum-free medium, plated on 0.1 mg/ml poly-D-lysine-coated tissue culture wells or plastic Lab-Tek slide wells, and maintained in a serum-free environment. The medium consisted of a 1:1 mixture of Eagle's MEM and Ham's F12 (Gibco, Gaithersburg, Md.) supplemented with glucose (6 mg/ml), putrescine (60 μM), progesterone (20 nM), transferrin (100 μg/ml), selenium (30 nM), penicillin (0.5 U/ml), and streptomycin (0.5 μg/ml) (Sigma, St. Louis, Mo.). In all experiments, neurons were cultured for 4-5 days before treatment. Cultures contained <2% glial cells, as confirmed by staining for glial markers.

### Example 4

### Immunocytochemistry

Immunocytochemistry for Examples 5-8 was performed in accordance with the following protocol. Cultured cells were fixed with 4% paraformaldehyde, exposed to primary antibodies at room temperature for 1.5 h, washed with PBS, exposed to the appropriate fluorescent secondary antibodies for 1 h at room temperature, exposed to Hoechst stain for 15 min at room temperature, and then analyzed with a Nikon fluorescent microscope. For uptake studies, living cultures were treated with FITC-siRNA, and analyzed with a Perkin-Elmer Spinning Disc confocal imaging system mounted on a Nikon inverted microscope.

### Example 5

### Uptake of Vector-Linked siRNA

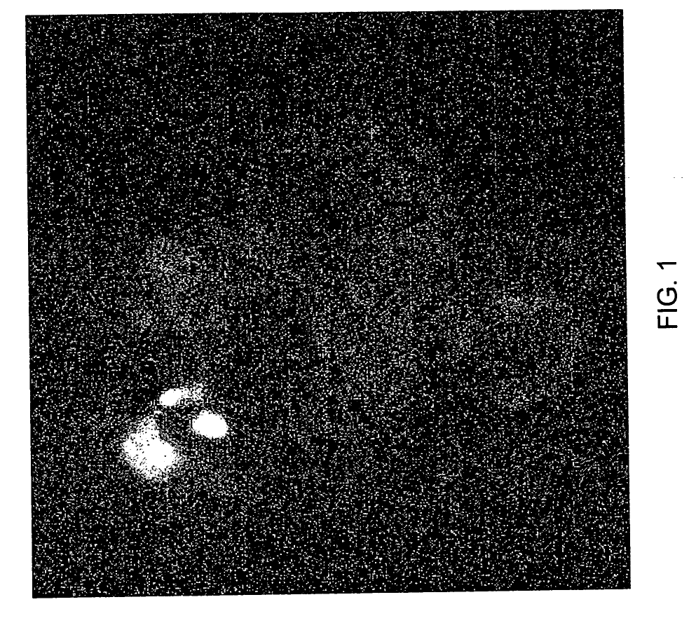

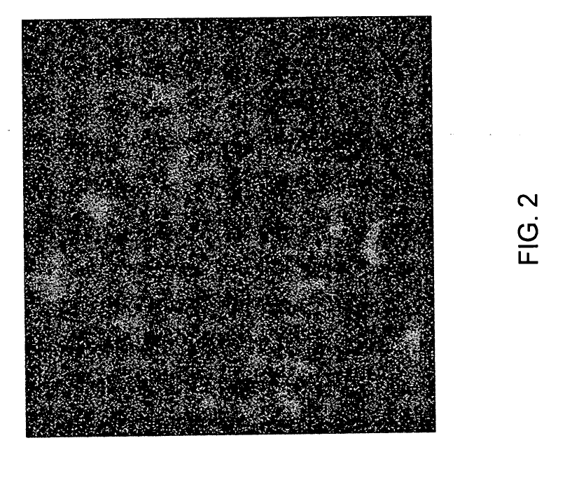

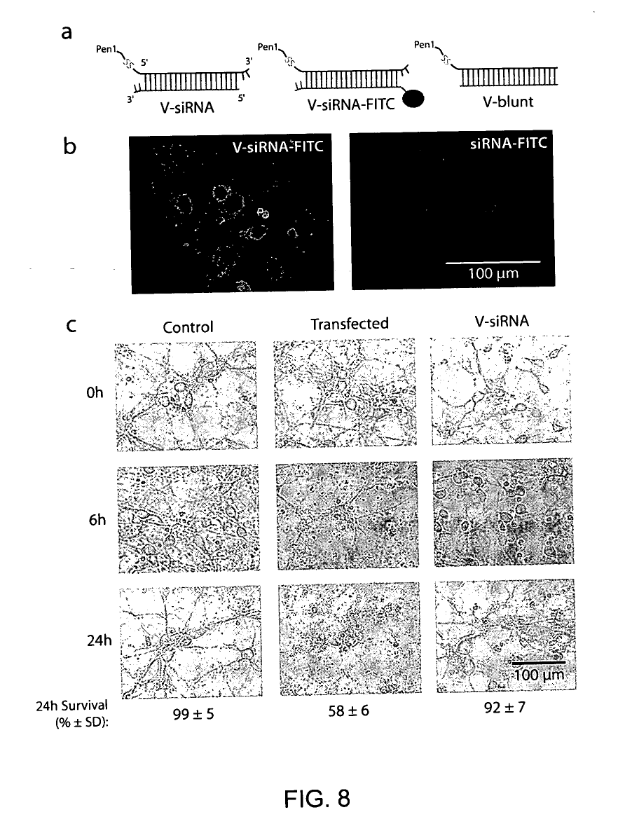

Transfection efficiencies of neuronal cells are generally low. To increase efficiency of delivery of small interfering ribonucleic acid (siRNA) to neuronal cells, the inventors designed siRNA molecules that could be linked to a cell-penetrating peptide. Specifically, either the sense or antisense strand of each small interfering RNA was modified at its 5′ end with a thiol group, and covalently bonded, via a disulfide bond, with a penetratin1 peptide having a pyridyl disulfide function at its terminal end. siRNA labelled with FITC was linked to the penetratin1 peptide, and applied to cultured rat sympathetic neurons prepared as described in Example 3. FITC was visualized with confocal microscopy. Uptake was rapid, within minutes of application of siRNA, as shown in FIG. 1 and FIG. 8B. Cultured hippocampal neurons were treated in the same way, and cultures were visualized 2 days later; the siRNA-FITC was still visible in the cytoplasm after 2 days, as shown in FIG. 2. Thus, vector-linked siRNA was rapidly taken up by neurons, and remained there for at least 2 days.

### Example 6

### Inhibition in Sympathetic Neurons

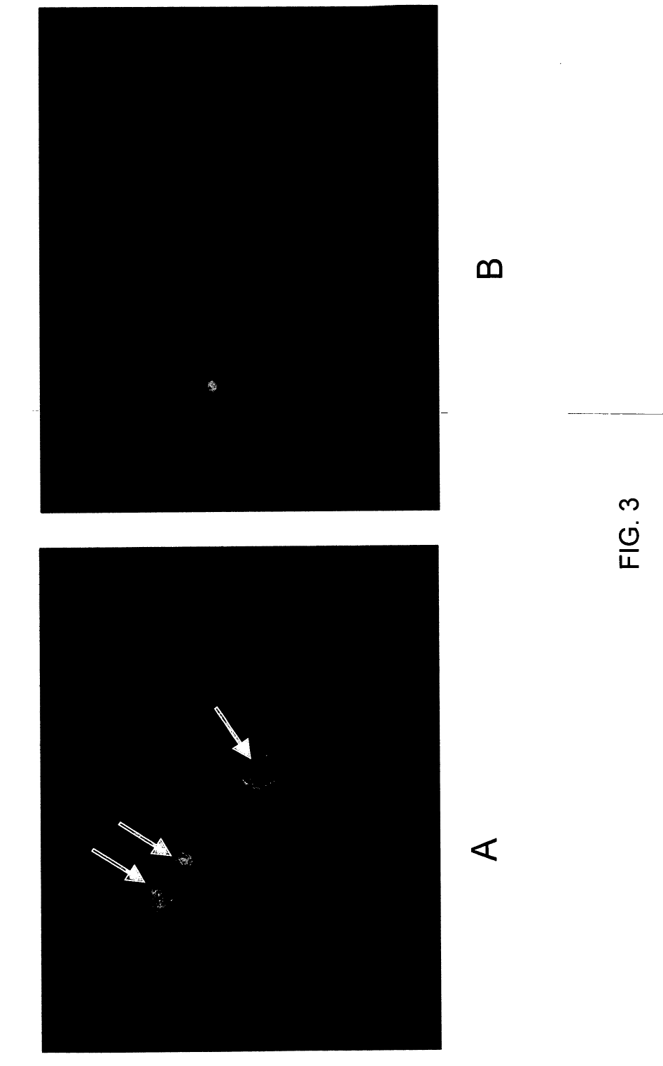

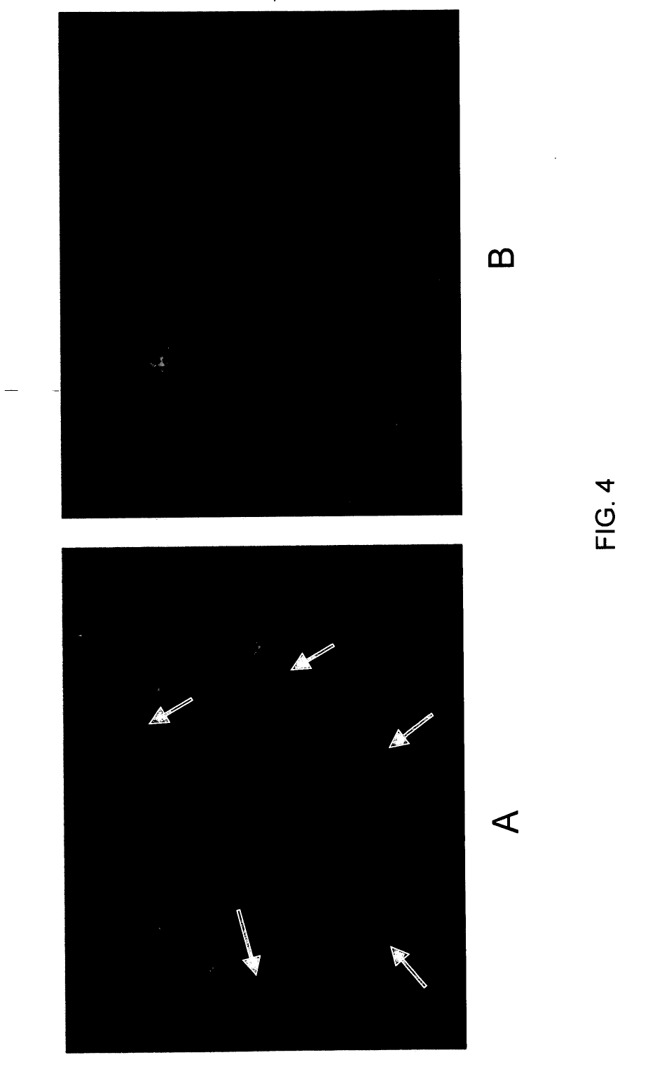

siRNAs were designed for two members of the caspase family of death proteases, caspase-8 and caspase-9, and linked to the penetratin1 peptide. Cultured mouse sympathetic neurons were treated with each of these constructs. Cultures were grown for one day, fixed and immunostained for caspase-8 or caspase-9 (together with Hoechst stain), and then visualized with fluorescent microscopy (FIGS. 3 and 4). Expression of the targeted caspase (caspase-8 or caspase-9) was inhibited in all of the cultured cells. Expression of non-targeted caspases was not changed. Therefore, vector-linked siRNA inhibited expression of specific proteins in sympathetic neurons.

### Example 7

### siRNA Targeted to SOD1

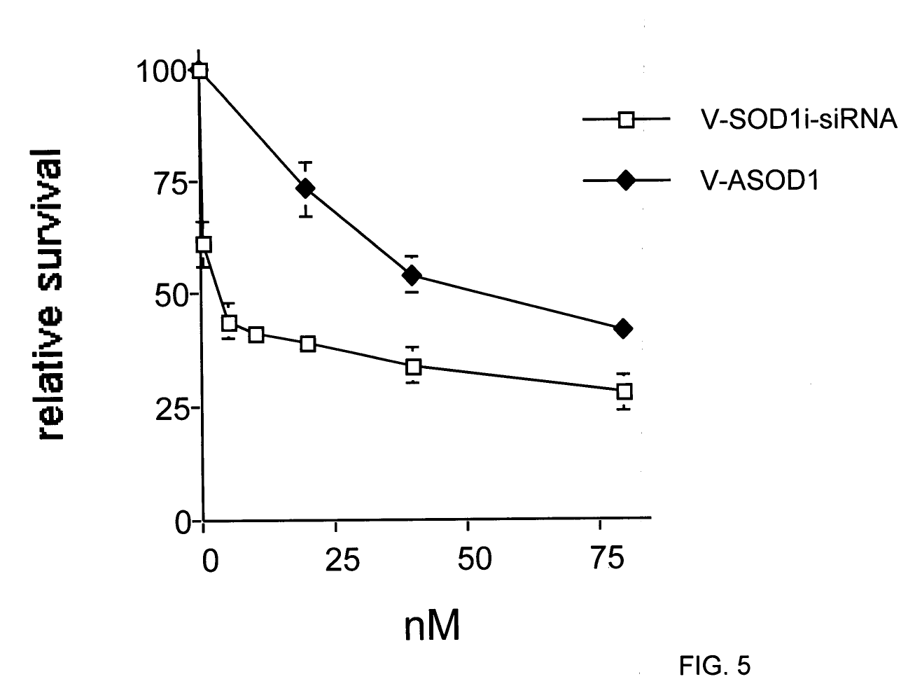

The inventors have previously shown that antisense oligonucleotides to SOD1 can down-regulate SOD1-specific activity in a dose-dependent manner (Troy et al., Down-regulation of Cu/Zn superoxide dismutase leads to cell death via the nitric oxide-peroxynitrite pathway, J. Neurosci., 16:253-61, 1996). This down-regulation induces cell death in cultured neurons. In the present study, the inventors determined that siRNA targeted to SOD1 could elicit the same effect. Cultured hippocampal neurons were treated with various concentrations of V-SOD1i (siRNA targeted to SOD1), and assayed for SOD activity. As shown in FIG. 5, there was a dose-dependent inhibition of SOD1-specific activity. Accordingly, vector-linked siRNA targeted to SOD1 inhibited SOD1-specific activity.

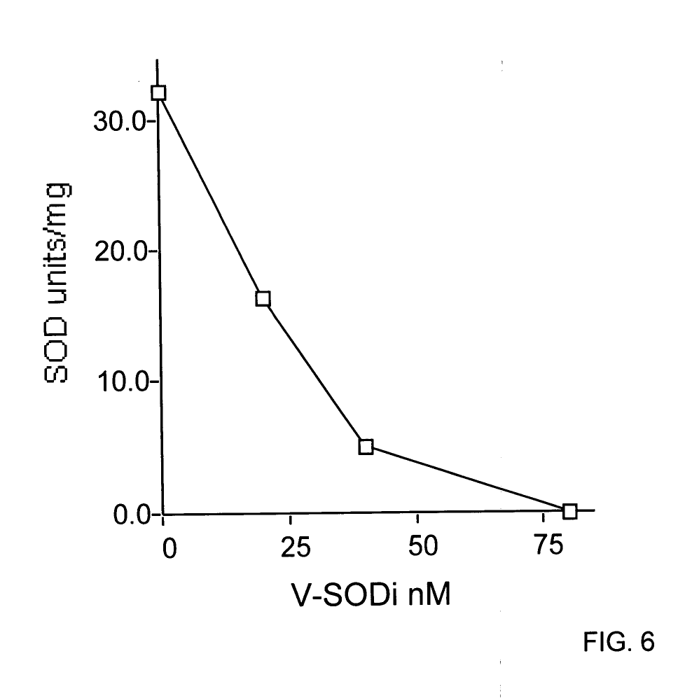

The inventors also compared the efficacy of the siRNA and the antisense oligonucleotide in inducing death in cultured neurons. As illustrated in FIG. 6, relative survival of hippocampal neurons, treated with either construct at the indicated concentrations, was determined. The siRNA was at least 10 times more potent in inducing death of the hippocampal neurons. siRNA that was unrelated to SOD1 did not affect the survival of hippocampal neurons. Thus, vector-linked siRNA targeted to SOD1 was more effective than vector-linked antisense oligonucleotide.

### Example 8

### Linkage of Vector to siRNA

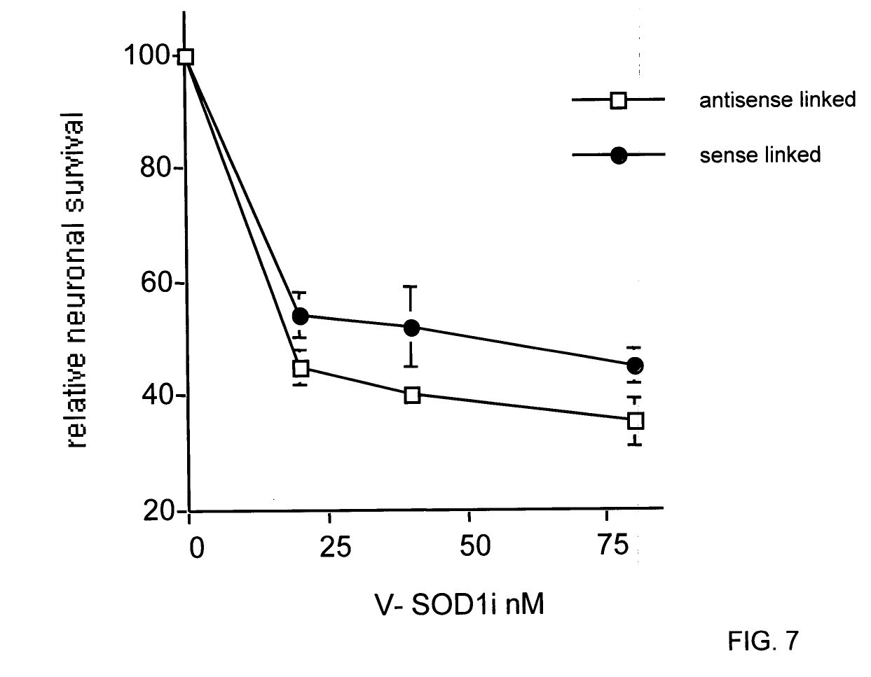

siRNA is double-stranded; either the sense or the antisense strand can be modified with the thiol group at the 5′ end, for linkage to the vector peptide. The inventors tested which strand was preferable using siRNA targeted to SOD1, and determined survival of hippocampal neurons treated with the indicated concentrations of each construct (FIG. 7). The constructs were equally effective in inducing death, suggesting that the vector peptide can be linked to either the sense or antisense strand of siRNA.

### Example 9

### Additional Cell Cultures

Cell cultures used in Examples 11-20 were prepared as follows. Primary cultures of dissociated sympathetic neurons were prepared from the superior cervical ganglia (SCG) of post-natal day 1 mice and rats (Troy et al., 2000; Troy et al., 2001). Briefly, SCGs were removed from the neonates, and capsules were removed by trypsinization. Cells were resuspended in RPMI, with 10% horse serum and mouse NGF (50 ng/ml), and plated at a density of 1 ganglion/well on Lab-Tek coverglass chamber slides coated with Matrigel. One day after plating, uridine and 5-fluorodeoxyuridine (10 μM each) were added to the cultures; the cultures were then left for 4 days, to eliminate non-neuronal cells. Hippocampal neuron cultures were prepared as described above, in Example 3.

### Example 10

### siRNA Design and Coupling

siRNAs for use in Examples 17-20 were prepared as follows. siRNAs were designed with dTdT 3′ overhangs. Sequences for the sense strand of the central 19-nt double-stranded region are set forth below:

SOD1i-1, UCCUCACUCUAAGAAACAU; (amino acid residues 3-21 of SEQ ID NO:1)
SOD1i-2, CCAGUGGUGGUGUCAGGAC; (amino acid residues 3-21 of SEQ ID NO:2)
Casp3i, CCUCAGAGAGACAUUCAUG; (SEQ ID NO:13)
Casp8i, GCACAGAGAGAAGAAUGAG; (amino acid residues 3-21 of SEQ ID NO:3)
Casp9i, GGCACCCUGGCUUCACUCU; (amino acid residues 3-21 of SEQ ID NO:5)
and
M2i, UCUUGUCAAAUUGGACAAAG. (SEQ ID NO:14)

siRNA duplexes, each having a 5′ thiol on the sense strand, were synthesized and HPLC purified (Dharmacon, Lafayette, Colo.; and Xeragon, Germantown, Md.). For uptake studies, siRNA was synthesized with a 5′ thiol on the sense strand, and a 5′ FITC on the antisense strand. Annealed siRNA duplexes were resuspended in buffer provided by the manufacturer, treated with an equimolar mixture of TCEP and an equimolar ratio of Penetratin1 (Q-Biogene, Carlsbad, Calif.), and incubated at 37° C. for 1 h. The yield of the reactions was estimated at 90% by SDS-PAGE, using SyProRuby (Molecular Probes, Eugene, Oreg.; data not shown).

### Example 11

### Transfection of Hippocampal Neurons

Lipofectamine 2000 was mixed with the siRNA construct, according to the manufacturer's instructions, and added to hippocampal neurons that had been in culture for 5 days.

### Example 12

### Immunocytochemistry

Immunocytochemistry for Examples 17-20 was performed in accordance with the following protocol. As described above, cells were grown and treated on 8-well Lab-Tek coverglass chamber slides, and fixed with 4% paraformaldehyde. After a 30-min block with 3% normal goat serum, slides were incubated with primary antibody for 1 h, and washed with PBS. Thereafter, the slides were incubated with appropriate secondary antibody (goat anti-rabbit or anti-mouse conjugated with rhodamine or FITC) for 30 min, followed by three PBS washes, and then incubated with Hoechst 33342 (1 μg/ml). Samples were visualized with a spinning disc confocal imaging system (Perkin-Elmer, Fremont, Calif.).

### Example 13

### Western-Blot Analysis

Hippocampal cultures were harvested in sample buffer. Equal amounts of protein were separated by 4-20% PAGE, transferred to nitrocellulose membrane, and immunostained as previously described (Troy, Rabacchi et al., 2001). Anti-SOD1 (Sigma, St. Louis, Mo.) was used at 1:300, anti-caspase-3 (United Biomedical, Inc., Hauppague, N.Y.) was used at 1:1000, anti-actin was used at 1:1000, and anti-cIAP2 (MBL International, Woburn, Mass.) was used at 1:1000. Visualization was achieved with ECL (Pierce, Rockford, Ill.).

### Example 14

### SOD1 Enzyme Activity

Hippocampal cultures were extracted with 0.5% Nonidet NP-40, and protein was measured with the Bio-Rad reagent (Bio-Rad, Hercules, Calif.). SOD levels were measured, as previously described (Troy and Shelanski, 1994), with a modification of the xanthine-xanthine oxidase system in which the reduction of nitroblue tetrazolium (NBT) was measured at 560 nm in the presence and absence of KCN. SOD1 activity was determined from an SOD standard curve, and is reported herein as the KCN-sensitive activity.

### Example 15

### Cell-Survival Quantification

Following removal of the medium, cultured hippocampal cells were lysed in 200 μl of a solution that lyses the cell membrane, but leaves the nuclei intact, as described in Troy and Shelanski, 1994. Intact nuclei were then counted using a hemacytometer. Cell counts were performed in quadruplicate wells. Statistical significance was determined by analysis of variance with Bonferroni's post-hoc analysis.

### Example 16

### Quantitative PCR

Primers were designed to amplify a 300- to 400-base region of the targeted mRNA, which spanned the siRNA targeting site. RNA was isolated from cultured neurons using Trizol reagent, and cDNA was prepared by reverse transcriptase using SuperScript II and oligo-dT primer (Invitrogen, Carlsbad, Calif.). cDNAs were added to a reaction mix of PCR ready-to-go beads (Amersham Pharmaceuticals, Buckinghamshire, UK) and SYBR Green (Molecular Probes), together with appropriate primers at 0.5 μM each. Levels of transcripts were analyzed using the Cepheid SmartCycler (Fisher, Pittsburgh, Pa.), in accordance with the manufacturer's specifications. For each mRNA, quantification was made from the linear portion of the amplification curve. Alpha-tubulin was used to normalize input cDNA.

### Example 17

### Uptake of Vector-Linked siRNA

The inventors have shown that vector-linked siRNA is rapidly and efficiently taken up by, and is non-toxic to, cultured primary neurons. To facilitate a determination as to whether vector-linked siRNA (V-siRNA) could be efficiently delivered to the cytoplasm as an intact duplex, the inventors synthesized an siRNA with a fluorescein group at the 5′ end of the antisense strand and a thiol group on the 5′ end of the sense strand. This siRNA was linked to penetratin1 (Pen1) by a disulfide bond (V-siRNA-FITC; FIG. 8A). The inventors monitored uptake by confocal microscopy, using the DNA-binding dye, DRAQ5, to visualize all cells in the live culture. Significant uptake of V-siRNA-FITC was observed within 15 min. At 2 h post-treatment, the medium was replaced with fresh medium; thereafter, V-siRNA-FITC treatment resulted in extensive punctate cytoplasmic fluorescence in greater than 95% of primary rat hippocampal neurons. Unlinked FITC-labelled siRNA (siRNA-FITC) was not taken up by the neurons (FIG. 8B). In all remaining experiments, Pen1 was linked to the siRNA by a thiol group on the 5′ end of the sense strand, while the antisense strand of the siRNA was unmodified.

Cell damage and death in complex heterogeneous cultures can lead to an alteration in the composition and function of primary neuronal cultures. Therefore, the inventors compared the cell-toxicity of vector-linked siRNAs with that of the transfection reagent, Lipofectamine 2000, which had been successfully used to deliver siRNA to primary neuronal cultures (Krichevsky and Kosik, RNAi functions in cultured mammalian neurons. Proc. Natl. Acad. Sci. USA, 99(18): 11926-9, 2002; Omi Tokunaga Hohjoh, Long-lasting RNAi activity in mammalian neurons. FEBS, 558(1-3):89-95, 2004; Fink et al., Selective regulation of neurite extension and synapse formation by the beta but not the alpha isoform of similar dependence on concentration of caspase-3-targeted siRNA (V-Casp3i), with 320 nM knocking protein down to 3% of control.

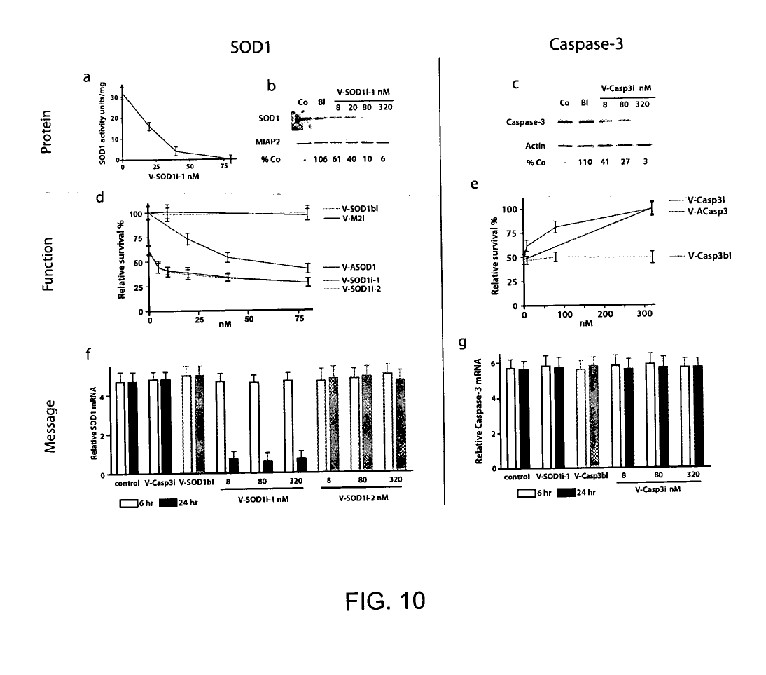

To confirm that the observed protein knockdown was functionally relevant, the inventors replicated earlier studies in which antisense oligonucleotides were delivered to neurons, either to protect from, or induce, apoptosis. Knockdown of SOD1 is known to induce an interleukin-1-dependent, nitric-oxide-mediated apoptosis (Troy, 1996). The inventors reveal herein that application of V-SOD1i-1 or V-SOD1i-2 to hippocampal neurons leads to a dose-dependent apoptotic death. At all concentrations, this effect is stronger for the V-siRNAs than for a vector-linked antisense oligonucleotide targeting SOD1 (V-ASOD1) (FIG. 10D).

Caspase-3 is a critical effector of 4-hydroxynonenal (HNE) death, and knockdown of caspase-3 protects neurons from HNE (Rabacchi et al., Divergence of the apoptotic pathways induced by 4-hydroxynonenal and amyloid beta-protein. Neurobiol. Aging. 25(8): 1057-66, 2004). In the present study, V-Casp3i was able to rescue hippocampal neurons from HNE-induced apoptosis in a dose-dependent fashion (FIG. 10E).

### Example 19

### Protein Knockdown can Precede mRNA Degradation

The inventors have also shown that siRNA-mediated protein knockdown does not require, and can precede, mRNA degradation. Given that RNAi in mammals is thought to result from the sequence-specific cleavage of homologous mRNA, the inventors examined the effect of V-siRNA on the levels of targeted mRNA. Total RNA was harvested from cultures, and real-time RT-PCR was performed using primers that spanned the regions targeted by the inventors' siRNAs. Surprisingly, at timepoints corresponding to nearly-complete down-regulation of targeted proteins, corresponding mRNA levels were unchanged.

For example, SOD1 protein knockdown at 6 h was confirmed by immunocytochemistry, Western blotting, and enzyme activity assays; mRNA levels at 6 h were unchanged, even by treatment at high concentrations of V-SOD1i-1. By 24 h post-treatment, however, SOD1 mRNA levels had declined by more than 80%. In contrast to the effect on protein observed at 6 h, there was no significant difference in mRNA levels at concentrations of 8, 80, and 320 nM, suggesting that the mechanism responsible for mRNA degradation was saturated at even the lowest concentration. CaMKII. Neuron, 39(2):283-97, 2003). Hippocampal cultures were evenly plated, and cultured for 5 days before treatment. Cells were imaged by phase microscopy, at 0, 6, 24, and 48 h post-treatment, and counted at 0, 24, and 48 h (FIG. 8C).

Cells in the transfected cultures showed evidence of injury. For example, by 6 h, there was substantial debris in the medium, neurites were beginning to degenerate, and some neuronal cell bodies had vacuoles; these were not seen in the control or V-siRNA-treated cultures. At 24 h post-treatment, there were very few neurites, and even fewer neurons, present in the transfected cultures. Cells treated with V-siRNA were qualitatively indistinguishable from untreated controls at all timepoints. Survival at 24 h, relative to the control at 0 h, was 99% for control, 92% for V-siRNA-treated cells, and 58% for cells transfected with Lipofectamine 2000 (FIG. 8C). At 48 h post-treatment, additional cell death was not observed, consistent with an acute insult due to the transfection. By 48 h, however, transfected cells had not regrown their extensive network of processes. All experiments were conducted in quadruplicate.

### Example 18

### Knockdown of Proteins

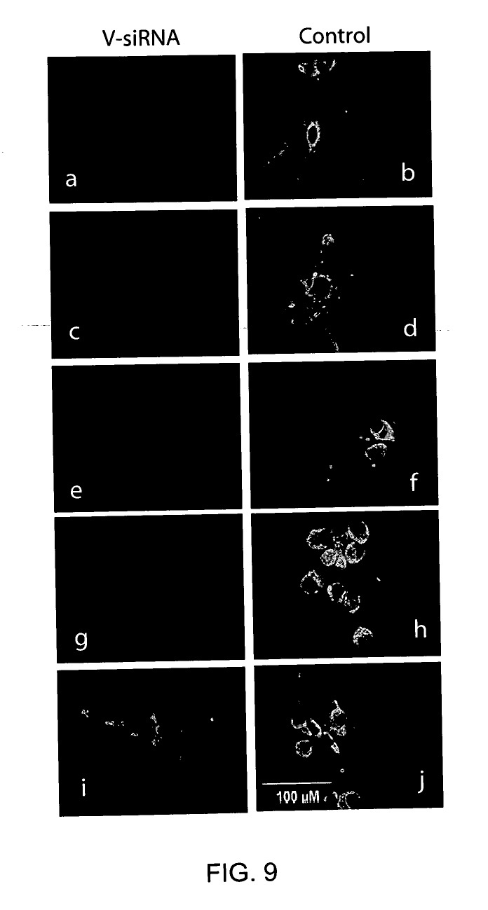

The inventors have demonstrated that V-siRNA knocks down levels of targeted endogenous proteins within hours. Immunocytochemistry revealed that treatment with targeted V-siRNA, at a concentration of 80 nM, was effective in knocking down levels of several endogenous proteins, at 5-6 h post-treatment, in nearly all cells in culture (FIG. 9). Hippocampal (FIGS. 9A-D) and sympathetic (FIGS. 9E-J) cultures were treated with the indicated V-siRNA for 6 h, and then fixed and immunostained for the target protein and a non-targeted protein. In all cases, the targeted protein (green) was down-regulated, and the non-targeted protein (red) was not. Two siRNAs, targeting Cu—Zn superoxide-dismutase-1 (SOD1), were both effective. Targeting of a caspase (e.g., caspases-3, -8, and -9) did not affect other, non-targeted, caspase family members.

Next, the inventors quantified the extent of knockdown of selected proteins at 6 h post-treatment, using a range of concentrations of V-siRNA. SOD1 levels were measured by an enzyme-activity assay, and by Western blotting (FIGS. 10A-B). By both measures, the knockdown in response to treatment with vector-linked siRNA targeting SOD1 (V-SOD1i-1) was dose-dependent, and nearly complete, at 80 nM (protein=10% of control, enzyme activity=0). Measurement of levels of caspase-3 protein, using Western blotting, showed a similar dependence on concentration of caspase-3-targeted siRNA (V-Casp3i), with 320 nM knocking protein down to 3% of control.

To confirm that the observed protein knockdown was functionally relevant, the inventors replicated earlier studies in which antisense oligonucleotides were delivered to neurons, either to protect from, or induce, apoptosis. Knockdown of SOD1 is known to induce an interleukin-1-dependent, nitric-oxide-mediated apoptosis (Troy, 1996). The inventors reveal herein that application of V-SOD1 i-1 or V-SOD1 i-2 to hippocampal neurons leads to a dose-dependent apoptotic death. At all concentrations, this effect is stronger for the V-siRNAs than for a vector-linked antisense oligonucleotide targeting SOD1 (V-ASOD1) (FIG. 10D).

Caspase-3 is a critical effector of 4-hydroxynonenal (HNE) death, and kockdown of caspase-3 protects neurons from HNE (Rabacchi et al., Divergence of the apoptotic pathways induced by 4-hydroxynonenal and amyloid beta-protein. Neurobiol. Aging. 25(8):1057-66, 2004). In the present study, V-Casp3i was able to rescue hippocampal neurons from HNE-induced apoptosis in a dose-dependent fashion (FIG. 10E).

### Example 19

### Protein Knockdown can Precede mRNA Degradation

The inventors have also shown that siRNA-mediated protein knockdown does not require, and can precede, mRNA degradation. Given that RNAi in mammals is thought to result from the sequence-specific cleavage of homologous mRNA, the inventors examined the effect of V-siRNA on the levels of targeted mRNA. Total RNA was harvested from cultures, and real-time RT-PCR was performed using primers that spanned the regions targeted by the inventors' siRNAs. Surprisingly, at timepoints corresponding to nearly-complete down-regulation of targeted proteins, corresponding mRNA levels were unchanged.

For example, SOD1 protein knockdown at 6 h was confirmed by immunocytochemistry, Western blotting, and enzyme activity assays; mRNA levels at 6 h were unchanged, even by treatment at high concentrations of V-SOD1 i-1. By 24 h post-treatment, however, SOD1 mRNA levels had declined by more than 80%. In contrast to the effect on protein observed at 6 h, there was no significant difference in mRNA levels at concentrations of 8, 80, and 320 nM, suggesting that the mechanism responsible for mRNA degradation was saturated at even the lowest concentration.

V-SOD1i-2, another siRNA targeting SOD1, showed knockdown of protein expression, and effectively induced hippocampal death, but did not alter mRNA levels of SOD1 at 6 or 24 h (FIG. 10F). Levels of mRNA unrelated to SOD1 were not altered (data not shown). V-Casp3i did not down-regulate caspase-3 mRNA at 6 or 24 h, although protein was knocked down by 6 h, and functional effects were apparent at 24 h (FIG. 10G).

### Example 20

### Requirement of 3′ 2-Nt Overhang

The inventors have also demonstrated that both early mRNA-like protein knockdown and siRNA-mediated mRNA degradation require a 3′ 2-nt overhang. In this regard, the inventors considered whether the early protein knockdown that they observed could be due to an antisense effect. Since siRNA is linked to Pen1 by its sense strand, the inventors knew that all antisense strands that reach the cytoplasm are delivered as part of intact duplexes. Furthermore, the inventors' functional results showed that, consistent with the first observations of RNAi (Fire and Mello, 1998), V-siRNAs were effective at much lower concentrations than were vector-linked antisense oligonucleotides.

The inventors determined that incorporation into the RNA-induced silencing complex (RISC) and micro-RNA-associated ribonucleoprotein complexes (miRNP) requires a 2-nt 3′ overhang on both ends of the siRNA. No such requirement exists for antisense oligonucleotides, which are delivered to the cell as single-stranded DNA or RNA, and are thought to inhibit translation by steric hindrance or RNaseH activation.

The inventors synthesized vector-linked RNA duplexes that had the same sequences as V-SOD1i-1 and V-Casp3i, over the central double-stranded portion of the siRNA, but lacked the 2-nt 3′ overhang (V-blunt; FIG. 8A). Treatment with V-blunt in the same concentrations as V-siRNA had no effect on either protein or message, and did not have functional effects (FIG. 10), suggesting that the observed early protein knockdown by perfectly homologous siRNAs was due to an mRNA-like translational suppression.

All publications, patent applications, and issued patents cited in this specification are herein incorporated by reference as if each individual publication, patent application, or issued patent was specifically and individually indicated to be incorporated by reference. Furthermore, the earlier incorporation by reference of any specific publication, patent application, or issued patent shall not negate this paragraph. The citation of any publication, patent application, or issued patent is made for its disclosure prior to the filing date of the subject application, and should not be construed as an admission that the present invention is not entitled to antedate such disclosure by virtue of prior invention.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A gene-silencing system comprising a ribonucleic acid (RNA) molecule linked to a cell-penetrating peptide, wherein the system is capable of silencing expression of a gene in a cell.

2. The system of claim 1, wherein the RNA molecule is modified for linkage with the cell-penetrating peptide.

3. The system of claim 2, wherein the modified RNA molecule is linked to the cell-penetrating peptide by a disulfide bond.

4. The system of claim 1, wherein the RNA molecule is single-stranded or double-stranded.

5. The system of claim 4, wherein the single-stranded RNA molecule has a hairpin structure.

6. The system of claim 4, wherein the single-stranded RNA molecule is a micro-RNA (mRNA) or a precursor thereof.

7. The system of claim 4, wherein the double-stranded RNA molecule is a small interfering RNA (siRNA).

8. The system of claim 4, wherein a strand of the double-stranded RNA molecule is modified at the 5′ end for linkage with the cell-penetrating peptide.

9. The system of claim 8, wherein the 5′ end of the strand is modified with a thiol group.

10. The system of claim 8, wherein a covalent bond links the modified 5′ end of the strand to the cell-penetrating peptide.

11. The system of claim 10, wherein the covalent bond is a disulfide bond.

12. The system of claim 1, wherein the RNA molecule specifically binds to mRNA transcribed from the gene.

13. The system of claim 12, wherein the RNA molecule is substantially homologous to or complementary to a portion of the transcribed mRNA.

14. The system of claim 13, wherein the gene is a human gene selected from the group consisting of a Cu—Zn superoxide dismutase-1 (SOD1) gene, a caspase 3 (Casp3) gene, a caspase 8 (Casp8) gene, and a caspase 9 (Casp9) gene.

15. The system of claim 13, wherein the RNA molecule comprises a nucleotide sequence selected from the group consisting of SEQ ID NO:1, SEQ ID NO:2, SEQ ID NO:3, SEQ ID NO:4, SEQ ID NO:5, SEQ ID NO:13, and SEQ ID NO:14.

16. The system of claim 1, wherein the cell-penetrating peptide is selected from the group consisting of penetratin, transportan, pIsl, TAT, pVEC, MTS, and MAP.

17. The system of claim 16, wherein the cell-penetrating peptide is penetratin.

18. The system of claim 1, wherein the RNA molecule is released from the cell-penetrating peptide inside the cell.

19. The system of claim 1, wherein expression of the gene is silenced at the protein and/or RNA level.

20. The system of claim 19, wherein expression of the gene is silenced only at the protein level.

21. The system of claim 19, wherein expression of the gene is silenced at both the protein and RNA levels, and wherein silencing at the protein level is followed by silencing at the RNA level.

22. The system of claim 1, wherein the cell is a post-mitotic cell.

23. The system of claim 22, wherein the cell is a neuron.

24. The system of claim 1, wherein the cell is a cell of a primary culture.

25. The system of claim 1, wherein the system has essentially no RNA-independent cytotoxicity.

26. The system of claim 1, further comprising at least one label affixed to the RNA molecule.

27. The system of claim 26, wherein the at least one label is affixed to the 5′ end of a strand of the RNA molecule.

28. The system of claim 26, wherein the label is an enzyme label, a chemical label, or a radioactive label.

29. The system of claim 1, further comprising a moiety conferring target-cell specificity to the system.

30. A pharmaceutical composition, comprising the system of claim 1 and a pharmaceutically-acceptable carrier, excipient, or diluent.

31. A cell comprising the system of claim 1.

32. The cell of claim 31, which is a post-mitotic cell.

33. The cell of claim 32, which is a neuron.

34. The cell of claim 31, which is a cell of a primary culture.

35. The cell of claim 31, wherein the system silences expression of the gene in the cell.

36. A kit for silencing expression of a gene in a cell, comprising:
(a) the system of claim 1; and
(b) optionally, instructions for using the system.

37. Use of the system of claim 1 in a method of studying protein function in one or more cells.

38. Use of the system of claim 1 in a method of studying interactions between neurons in culture.

39. A culture of primary cells, wherein the cells have been contacted with at least one gene-silencing system comprising a ribonucleic acid (RNA) molecule linked to a cell-penetrating peptide, and wherein the at least one system is capable of silencing expression of a gene in the cells.

40. The culture of claim 39, wherein the cells are mammalian cells.

41. The culture of claim 40, wherein the cells are neurons.

42. The culture of claim 41, wherein the neurons are hippocampal neurons.

43. The culture of claim 39, wherein the RNA molecule is labelled with a fluorescent dye.

44. The culture of claim 43, wherein the fluorescent dye is fluorescein.

45. The culture of claim 39, wherein the at least one system silences expression of the gene in one or more of the cells.

46. A gene-silencing system comprising:
(a) a small interfering RNA (siRNA) molecule comprising a duplex region of at least 19 nucleotides, wherein at least one strand of the duplex region is homologous to a portion of mRNA transcribed from a gene, and wherein a strand of the siRNA molecule is modified at the 5′ end for linkage with a cell-penetrating peptide;
(b) a cell-penetrating peptide selected from the group consisting of penetratin, transportan, pIsl, TAT, pVEC, MTS, and MAP; and
(c) a covalent bond linking the siRNA molecule to the cell-penetrating peptide;
wherein the system is capable of silencing expression of the gene in a cell.

47. A pharmaceutical composition, comprising the system of claim 46 and a pharmaceutically-acceptable carrier, excipient, or diluent.

48. A cell comprising the system of claim 46.

49. A kit for silencing expression of a gene in a cell, comprising:
(a) the system of claim 46; and
(b) optionally, instructions for using the system.

50. Use of the system of claim 46 in a method selected from the group consisting of a method of studying protein function in one or more cells and a method of studying interactions between neurons in culture.

51. A system for use in genetic screening, comprising a plurality of RNA molecules, wherein each RNA molecule is linked to a cell-penetrating peptide.

52. The system of claim 51, wherein at least one of the RNA molecules, when contacted with a cell, has an effect on at least one cellular event in the cell.

53. The system of claim 52, wherein the at least one cellular event in the cell results in at least one detectable change in phenotype of the cell.

54. The system of claim 51, further comprising at least one label affixed to at least one of the RNA molecules.

55. A method for silencing expression of a gene in a cell, comprising contacting a cell with a gene-silencing system, wherein the system comprises a ribonucleic acid (RNA) molecule linked to a cell-penetrating peptide, and wherein the system is capable of silencing expression of a gene in the cell.

56. The method of claim 55, wherein expression of the gene is silenced at the protein and/or RNA level.

57. The method of claim 56, wherein expression of the gene is silenced only at the protein level.

58. The method of claim 56, wherein expression of the gene is silenced at both the protein and RNA levels, and wherein silencing at the protein level is followed by silencing at the RNA level.

59. The method of claim 55, wherein the RNA molecule is released from the cell-penetrating peptide inside the cell.

60. The method of claim 55, wherein the cell is a mammalian cell.

61. The method of claim 60, wherein the mammalian cell is a human cell.

62. The method of claim 60, wherein the mammalian cell is a neuron.

63. The method of claim 55, wherein the cell is in a tissue.

64. The method of claim 55, wherein the cell is contacted with the gene-silencing system ex vivo.

65. The method of claim 55, wherein the cell is contacted with the gene-silencing system in vivo in a subject.

66. The method of claim 65, wherein the cell is contacted with the gene-silencing system in vivo in a subject via intradermal, oral, parenteral, rectal, in situ, topical, and/or transdermal administration to the subject.

67. A method for silencing expression of a gene in a cell, comprising contacting a cell with an amount of a gene-silencing system effective to silence expression of a gene in the cell, wherein the gene-silencing system comprises:
(a) a small interfering RNA (siRNA) molecule comprising a duplex region of at least 19 nucleotides, wherein at least one strand of the duplex region is homologous to a portion of mRNA transcribed from the gene, and wherein a strand of the siRNA molecule is modified at the 5′ end for linkage with a cell-penetrating peptide;
(b) a cell-penetrating peptide selected from the group consisting of penetratin, transportan, pIsl, TAT, pVEC, MTS, and MAP; and
(c) a covalent bond linking the siRNA molecule to the cell-penetrating peptide;
wherein the system is capable of silencing expression of the gene in the cell.

68. A method for performing genetic screening, comprising the steps of:
(a) providing a collection of cells;
(b) contacting the cells with a system comprising a plurality of RNA molecules, wherein each RNA molecule is linked to a cell-penetrating peptide;
(c) determining whether one or more of the RNA molecules cause one or more detectable changes in phenotype of the cells; and
(d) identifying one or more genes or proteins responsible for the one or more changes in phenotype of the cells.

69. A method for determining the function of a gene in a cell, comprising the steps of:
(a) providing a gene-silencing system comprising a ribonucleic acid (RNA) molecule linked to a cell-penetrating peptide, wherein the system is capable of silencing expression of a gene in a cell;
(b) contacting a cell with the gene-silencing system, such that expression of a gene is silenced in the cell;
(c) assessing the phenotype of the cell resulting from step (b); and
(d) comparing the phenotype of the cell in step (c) to that of an appropriate control cell, thereby determining the function of the gene in the cell.

70. A method for determining the function of a gene in a cell, comprising the steps of:
(a) providing a gene-silencing system comprising:
(i) a small interfering RNA (siRNA) molecule comprising a duplex region of at least 19 nucleotides, wherein at least one strand of the duplex region is homologous to a portion of mRNA transcribed from a gene, and wherein a strand of the siRNA molecule is modified at the 5′ end for linkage with a cell-penetrating peptide;
(ii) a cell-penetrating peptide selected from the group consisting of penetratin, transportan, pIsl, TAT, pVEC, MTS, and MAP; and
(iii) a covalent bond linking the siRNA molecule to the cell-penetrating peptide;
wherein the system is capable of silencing expression of the gene in the cell;
(b) contacting the cell with the gene-silencing system, such that expression of the gene is silenced in the cell;
(c) assessing the phenotype of the cell resulting from step (b); and
(d) comparing the phenotype of the cell in step (c) to that of an appropriate control cell, thereby determining the function of the gene in the cell.


SEQUENCE LISTING (18 sequences)

SEQ ID NO: 1 - RNA, 21 nt, Homo sapiens
  misc_RNA (0)...(0)
aauccucacucuaagaaacau

SEQ ID NO: 2 - RNA, 21 nt, Homo sapiens
aaccagugguggugucaggac

SEQ ID NO: 3 - RNA, 21 nt, Homo sapiens
  misc_RNA (0)...(0)
aagcacagagagaagaaugag

SEQ ID NO: 4 - RNA, 21 nt, Homo sapiens
  misc_RNA (0)...(0)
aagaagcaggagaccaucgag

SEQ ID NO: 5 - RNA, 21 nt, Mus musculis
  misc_RNA (0)...(0)
aaggcacccuggcuucacucu

SEQ ID NO: 6 - protein, 16 aa, Drosophila antennapedia
  PEPTIDE (0)...(0)
RQIKIWFQNRRMKWKK

SEQ ID NO: 7 - protein, 27 aa, Artificial Sequence
  synthetic peptide
GWTLNSAGYLLGKINLKALAALAKKIL

SEQ ID NO: 8 - protein, 16 aa, Rattus sp.
  PEPTIDE (0)...(0)
PVIRVWFQNKRCKDKK

SEQ ID NO: 9 - protein, 13 aa, Human immunodeficiency virus type 1
GRKKRRQRRRPPQ

SEQ ID NO: 10 - protein, 16 aa, Mus musculis
  PEPTIDE (0)...(0)
LLIILRRRIRKQAHAH

SEQ ID NO: 11 - protein, 27 aa, Artificial Sequence
  MPS peptide is a chimera of the hydrophobic terminal domain of the viral gp41 protein and the nuclear localization signal from simian virus 40 large antigen.
GALFLGWLGAAGSTMGAWSQPKKKRKV

SEQ ID NO: 12 - protein, 19 aa, Artificial Sequence
  synthetic alpha-helical amphipathic model peptide
KLALKLALKALKAALKLAX

SEQ ID NO: 13 - RNA, 19 nt, Artificial Sequence
  synthetic siRNA
ccucagagagacauucaug

SEQ ID NO: 14 - RNA, 20 nt, Artificial Sequence
  synthetic siRNA
ucuugucaaauuggacaaag

SEQ ID NO: 15 - RNA, 19 nt, Artificial Sequence
  synthetic siRNA
uccucacucuaagaaacau

SEQ ID NO: 16 - RNA, 19 nt, Artificial Sequence
  synthetic siRNA
ccagugguggugucaggac

SEQ ID NO: 17 - RNA, 19 nt, Artificial Sequence
  synthetic siRNA
gcacagagagaagaaugag

SEQ ID NO: 18 - RNA, 19 nt, Artificial Sequence
  synthetic siRNA
ggcacccuggcuucacucu